(19) United States
(12) Patent Application Publication
(10) Pub. No.: US 2024/0341020 A1
(43) Pub. Date: Oct. 10, 2024

(54) AEROSOL-GENERATING DEVICE

(71) Applicant: Philip Morris Products S.A., Neuchatel (CH)

(72) Inventors: Enrico STURA, Palezieux-Village (CH); Torsten Richard Mathias TINGSTRÖM, Morges (CH); Ezequiel Martin VALDEZ ROJAS, Pully (CH); Valerio OLIANA, Pully (CH)

(73) Assignee: Philip Morris Products S.A., Neuchatel (CH)

(21) Appl. No.: 18/573,135

(22) PCT Filed: Jun. 27, 2022
(86) PCT No.: PCT/EP2022/067604

§ 371 Date: Dec. 21, 2023

(30) Foreign Application Priority Data
Jun. 30, 2021 (EP) 21182925.4

(51) Int. Cl. H05B 47/105; A24F 40/20; A24F 40/53; A24F 40/57; A24F 40/60; F21V 23/00; F21Y 105/16; F21Y 113/13; F21Y 115/10; H05B 45/20

(52) U.S. Cl. CPC H05B 47/105; A24F 40/53; F21V 23/009; H05B 45/20; A24F 40/20; A24F 40/57; A24F 40/60; F21Y 2105/16; F21Y 2113/13; F21Y 2115/10

(57) ABSTRACT

An aerosol-generating device for heating an aerosol-forming substrate to generate an inhalable aerosol is provided, the device including: control electronics; and substantially linear first and second lighting arrays, each extending over a length between first and second ends of the respective array, the first and second arrays being a same length and laterally spaced apart from each other to be parallel and aligned with each other, and the first and second ends of the first array are aligned with the first and second ends of the second array, the control electronics being coupled to the first and second arrays to activate both of the first and second arrays to generate a predetermined light emission indicative of and in response to at least one of a status and progression of an operational phase of the device, and to cause each of the first and the second arrays to convey different data.

The present disclosure relates to an aerosol-generating device in which data concerning the progression of an operational phase of the device is visually conveyed to a user of the device.

Aerosol-generating devices configured to generate an aerosol from an aerosol-forming substrate, such as a tobacco containing substrate, are known in the art. Typically, an inhalable aerosol is generated by the transfer of heat from a heat source to a physically separate aerosol-forming substrate or material, which may be located within, around or downstream of the heat source. An aerosol-forming substrate may be a liquid substrate contained in a reservoir. An aerosol-forming substrate may be a solid substrate. An aerosol-forming substrate may be a component part of a separate aerosol-generating article configured to engage with an aerosol-generating device to form an aerosol. During consumption, volatile compounds are released from the aerosol-forming substrate by heat transfer from the heat source and entrained in air drawn through the aerosol-generating article. As the released compounds cool, they condense to form an aerosol that is inhaled by the consumer.

During use of the aerosol-generating device, changes in one or more parameters of the device may occur. It is desired to provide an aerosol-generating device which is able to efficiently convey data concerning the state of the device to a user.

As used herein, the term “aerosol-generating device” is used to describe a device that interacts with an aerosol-forming substrate of an aerosol-generating article to generate an aerosol. Preferably, the aerosol-generating device is a smoking device that interacts with an aerosol-forming substrate of an aerosol-generating article to generate an aerosol that is directly inhalable into a user's lungs thorough the user's mouth. The aerosol-generating device may be a holder for a smoking article. Preferably, the aerosol-generating article is a smoking article that generates an aerosol that is directly inhalable into a user's lungs through the user's mouth. More preferably, the aerosol-generating article is a smoking article that generates a nicotine-containing aerosol that is directly inhalable into a user's lungs through the user's mouth.

As used herein, the term “aerosol-forming substrate” denotes a substrate consisting of or comprising an aerosol-forming material that is capable of releasing volatile compounds upon heating to generate an aerosol.

According to an aspect of the present invention, there is provided an aerosol-generating device for heating an aerosol-forming substrate to generate an inhalable aerosol during a usage session. The aerosol-generating device comprises: control electronics; a substantially linear first lighting array and a substantially linear second lighting array. Each of the first and second lighting arrays extend over a length between first and second ends of the respective lighting array. The control electronics are coupled to the first and second lighting arrays and configured to activate either or both of the first and second lighting arrays to generate a predetermined light emission indicative of and in response to at least one of: i) a status of the aerosol-generating device; and ii) progression of an operational phase of the aerosol-generating device.

As used herein, the term “light” refers to emissions of electromagnetic radiation which are in the visible range of the electromagnetic spectrum. The visible range of the electromagnetic spectrum is generally understood to encompass wavelengths in a range of about 380 nanometres to about 750 nanometres.

As used herein, the term “predetermined light emission” is an emission of light characterised in terms of one or more parameters of the light emission. By way of example, the one or more parameters may include any of: a luminance level of the light emission, a spatial variation in luminance level of the light emission over one or both of the first and second lighting arrays, a colour of the light emission, a spatial variation in colour of the light emission over one or both of the first and second lighting arrays, a proportion of one or both of the first and second lighting arrays which is activated to generate the light emission. The one or more parameters may also include a variation with time of any of the parameters described in the previous sentence.

The operational phase may be any phase of operation of the aerosol-generating device. By way of example and without limitation, the operational phase may be a pre-heating phase of operation or the usage session.

The pre-heating phase of operation is a phase of operation of the aerosol-generating device in which a temperature of an electrical heating arrangement of the aerosol-generating device is increased to a predetermined target temperature.

The usage session is a finite usage session; that is a usage session having a start and an end. The duration of the usage session as measured by time may be influenced by use during the usage session. The duration of the usage session may have a maximum duration determined by a maximum time from the start of the usage session. The duration of the usage session may be less than the maximum time if one or more monitored parameters reaches a predetermined threshold before the maximum time from the start of the usage session. By way of example, the one or more monitored parameters may comprise one or more of: i) a cumulative puff count of a series of puffs drawn by a user since the start of the usage session, and ii) a cumulative volume of aerosol evolved from the aerosol-forming substrate since the start of the usage session.

The coupling of the control electronics to the first and second lighting arrays as described above allows the lighting arrays to provide a user with data in a visual format indicative of the state of the device or of progression of the operational phase of the device. The use of two lighting arrays may allow each of the first and second lighting arrays to convey different data to a user. The linear nature of the first and second lighting arrays is especially suitable for tracking the progression of an operational phase of the aerosol-generating device through corresponding changes in the predetermined light emission. The changes in the predetermined light emission with progression of the operational phase may take the form of a change of one or more of: a luminance level of the light emission, a colour or distribution of colours forming the light emission, and the proportion of one or both of the first and second lighting arrays which is activated to generate the light emission.

Preferably, the control electronics may be configured to activate either or both of the first and second lighting arrays at two or more luminance levels, so as to control the luminance of the predetermined light emission. In this manner, the luminance level of the predetermined light emission may provide a user with an indication of the status of the aerosol-generating device, or of the progression through the operational phase. By way of example, where the predetermined light emission is indicative of progression through the pre-heating phase of operation or the usage session, the luminance level of one or both of the first and second lighting arrays may be increased or decreased when generating the predetermined light emission with progression through the pre-heating phase or usage session.

The control electronics may be configured to activate either or both of the first and second lighting arrays in two or more colour states, so as to control the colour of the predetermined light emission. In this manner, the colour of the predetermined light emission may provide a user with an indication of the status of the aerosol-generating device, or of the progression through the operational phase. The “colour” of the predetermined light emission may be a spatial variation of colour over one or both of the first and second lighting arrays.

The control electronics may be configured to activate either or both of the first and second lighting arrays so as to vary the predetermined light emission with respect to time. The variation of the predetermined light emission with respect to time may be particularly beneficial in enabling the control electronics to adjust the predetermined light emission so as to track and be indicative of changes in the state of the aerosol-generating device, or of the progression through the operational phase. The variation of the predetermined light emission with respect to time may be a variation of one or more of a luminance of the predetermined light emission, a colour of the predetermined light emission and a proportion of one or both of the lighting arrays which are activated to generate the predetermined light emission.

The control electronics may be configured to activate either or both of first and second lighting arrays to vary the predetermined light emission with respect to time so as to be indicative of the progression of the operational phase of the aerosol-generating device. Conveniently, the progression of the operational phase may be the progression of the usage session. Alternatively, the progression of the operational phase may be the progression of a pre-heating phase of operation of an electrical heating arrangement used to heat the aerosol-forming substrate.

The control electronics may be configured to activate either or both of the first and second lighting arrays so as to vary an activated length of the respective lighting array with respect to time. By varying the activated length of the respective lighting array, the proportion of the respective lighting array which contributes to generating the predetermined light emission is varied. The control electronics may be configured to increase or reduce an activated length of the respective lighting array whilst the aerosol-generating device remains in a given state, or with progression through the operational phase of the aerosol-generating device. This may be particularly beneficial where the operational phase is a pre-heating phase of operation of an electrical heating arrangement or the usage session, with the increase or decrease in the activated length efficiently conveying data to a user relating to the progression through the pre-heating phase or the usage session.

The control electronics may be configured to vary the predetermined light emission with respect to time in one or more of luminance and colour. An increase or decrease in luminance or colour with respect to time may be particularly beneficial in communicating to a user that a change has occurred in the temperature of an electrical heating arrangement of the aerosol-generating device. By way of example, as the temperature of the electrical heating arrangement is increased towards a predetermined target temperature, the predetermined light emission may be adjusted from a first state in which it consists of or comprises a colour closer to the blue end of the electromagnetic spectrum to a second state in which it consists of or comprises a colour closer to the red end of the electromagnetic spectrum.

The control electronics may be configured to vary the predetermined lighting emission with respect to time by one or more of activating, deactivating and reactivating different portions of either or both of the first and second lighting arrays over time. The change with respect to time as to which portion or portions of one or both of the first and second lighting arrays are activated may facilitate efficiently communicating data to a user indicating that there has been a change in state of the aerosol-generating device, or a change in the progression of an operational phase of the aerosol-generating device.

Preferably, each of the first and second lighting arrays comprise a plurality of light emitting units distributed between the first and second ends of the respective lighting array. So, each or different ones of the light emitting units may contribute towards the predetermined light emission according to which of the light emitting units is activated by the control electronics at a given instant in time. All or only some of the light emitting units may be used in the generating of the predetermined light emission at a given instant in time. The use of light emitting units in the form of light emitting diodes (LED's) is preferred due to LED's being energy efficient. It is preferred that the aerosol-generating device is sized so as to be handheld and to include a power source to provide portability. The power source may conveniently be in the form of a rechargeable battery. In this context, the energy efficiency associated with LED's makes them particularly suitable for use in such a handheld portable aerosol-generating device having its own power source. Alternatively however, the light emitting units may instead be comprised of one or more liquid crystal displays, or any other electrically powered light source whose energy and size requirements are suitable for use in an aerosol-generating device.

Conveniently, the aerosol-generating device further comprises one or more waveguides configured to direct light generated by one or more of the plurality of light emitting units to one or more display windows for viewing of the predetermined light emission by a user. As used herein, the term “waveguide” denotes a structure adapted to guide electromagnetic waves of light. The waveguide may conveniently be in the form of one or more optical fibres or light pipes. Conveniently, each of the light emitting units is associated with a corresponding waveguide, so that the light emitted from each light emitting unit is conveyed to the one or more display windows via the corresponding waveguide.

Preferably, each one of the plurality of light emitting units may comprise a light emitting diode and the control electronics may comprise a light emitting diode control driver and a separate microcontroller. The control driver may be configured to control a supply of electricity from a power source to one or more of the plurality of light emitting diodes of either or both of the first and second lighting arrays under the control of the microcontroller, so as to generate the predetermined light emission. The control driver may be configured to control one or both of the voltage or current level of the supply of electricity.

The plurality of light emitting diodes of each of the first and second lighting arrays may additionally comprise: a first set of one or more light emitting diodes configured to emit light of a first colour; and a second set of one or more light emitting diodes configured to emit light of a second colour. The light emitting diode control driver may be configured to activate one or more of the light emitting diodes from the first set alone of either or both of the first and second lighting arrays, or from the second set alone of either or both of the first and second lighting arrays, or from both of the first and second sets of either or both of the first and second lighting arrays, so as to control the colour of the predetermined light emission.

Conveniently, the light emitting diode control driver may be configured to control a supply of electricity from a power source to one or more of the plurality of light emitting diodes of either or both of the first and second lighting arrays by a pulse width modulation regime having a predetermined resolution, so as to control the luminance of the predetermined light emission, in which the predetermined resolution defines two or more luminance levels. By way of example, the resolution of the pulse width modulation regime may be 8 bit (having 256 levels), 10 bit (having 1024 levels), or 12 bit (having 4096 levels). The higher the predetermined resolution, the greater the number of discrete static luminance levels of light which may be generated by each one of the plurality of light emitting diodes. In this manner, the granularity or level of detail of data conveyed to the user through the different luminance levels may be controlled by the predetermined resolution chosen for the light emitting diode control driver.

Preferably, the length of the first lighting array may be the same as the length of the second lighting array.

Preferably, the first lighting array and the second lighting array may be laterally spaced apart from each other and parallel to each other.

Advantageously, the first and second lighting arrays are each of the same length and laterally spaced apart from each other so as to be parallel and aligned with each other, with the first and second ends of the first lighting array aligned with the first and second ends of the second lighting array.

Preferably, the predetermined light emission is one or more of a usage session light emission, a low energy light emission, a thermal profile light emission, a pause light emission, a state change light emission, a progressing light emission and a pre-heating light emission. By “usage session light emission” is meant a light emission indicative of a power source of the aerosol-generating device having containing sufficient energy for completing a predetermined number of usage sessions. By “low energy light emission” is meant a light emission indicative of a power source of the aerosol-generating device containing a level of energy less than or equal to a predetermined threshold level of energy. By “thermal profile light emission” is meant a light emission indicative of selection of one of at least two predetermined thermal profiles of an electrical heating arrangement of the aerosol-generating device. By “pause light emission” is mean a light emission indicative of the aerosol-generating device being in a pause mode. By “state change light emission” is meant a light emission indicative of a change in operational state of the aerosol-generating device. By “progressing light emission” is meant a light emission indicative of progression through the usage session. By “pre-heating light emission” is meant a light emission indicative of progression through a pre-heating phase of operation of an electrical heating arrangement of the aerosol-generating device. Examples relating to these different forms of “light emission” are outlined in the paragraphs below.

Although the above paragraphs describe an arrangement of first and second lighting arrays, the provision of additional lighting arrays is not excluded. In particular, advantageously, the aerosol-generating device may further a substantially linear third lighting array located between and parallel to each of the first and second lighting arrays, in which the control electronics are configured to activate the third lighting array alone or in addition to either or both of the first and second lighting arrays to generate the predetermined light emission. The addition of a third lighting array alongside the first and second lighting arrays further increases the complexity and granularity of data which may be conveyed to a user relating to the status and progression of an operational phase of the aerosol-generating device.

Conveniently, the aerosol-generating device may further comprise: a power source coupled to the control electronics. The control electronics may be configured to: determine a level of energy contained in the power source; and compare the determined level of energy with first and second predetermined energy thresholds. The first predetermined energy threshold may correspond to the power source containing sufficient energy to complete a single usage session. The second predetermined energy threshold may correspond to the power source containing sufficient energy to complete two or more usage sessions. The control electronics may also be configured to: activate either or both of the first and second lighting arrays to generate a single usage session light emission in response to a first state in which the determined level of energy is sufficient to complete a single usage session; and activate either or both of the first and second lighting arrays to generate a plural usage sessions light emission in response to a second state in which the determined level of energy is sufficient to complete two or more usage sessions. The single usage session light emission and the plural usage sessions light emission are different to each other. The single usage session light emission is indicative of the first state and the plural usage sessions light emission is indicative of the second state. In this manner, a user may be provided with a visual indication as to whether the power source has sufficient energy to complete either a single usage session or plural usage sessions. By way of example, the power source may be selected to have an energy capacity sufficient to complete two usage sessions before requiring replacement or recharging, with the plural usage sessions being two usage sessions. However, the energy capacity of the power source may be chosen to enable the completion of more than two usage sessions before requiring replacement or recharging.

The control electronics may be configured to activate a greater proportion of either or both of the first and second lighting arrays to generate the plural usage sessions light emission than to generate the single usage session light emission.

The control electronics may be configured to: activate all or part of only the first lighting array to generate the single usage session light emission in response to the first state; and activate all or part of both the first lighting array and the second lighting array to generate the plural usage sessions light emission in response to the second state. Conveniently, the control electronics may be configured to: activate between 90% to 100% of the length of the first lighting array to generate the single usage session light emission; and activate between 90% to 100% of the length of both the first lighting array and the second lighting array to generate the plural usage sessions light emission.

The control electronics may be configured to activate either or both of the first and second lighting arrays such that the single usage session light emission and the plural usage sessions light emission are different to each other in one or more of luminance and colour. Conveniently, the control electronics may be configured to activate either or both of the first and second lighting arrays such that the single usage session light emission has a first predetermined luminance and the plural usage sessions light emission has a second predetermined luminance, the second predetermined luminance being greater than the first predetermined luminance.

Conveniently, the aerosol-generating device may further comprise: a power source coupled to the control electronics. The control electronics may be configured to: determine a level of energy contained in the power source and compare the determined level of energy with a predetermined threshold level of energy; and activate either or both of the first and second lighting arrays to generate a low energy light emission in response to the determined level of energy being less than or equal to the predetermined threshold level of energy. The low energy light emission is indicative of the determined level of energy being less than or equal to the predetermined threshold level of energy. In this manner, a user may be provided with a visual indication of the power source having insufficient energy to complete a full usage session. Where the power source is a rechargeable power source, the low energy light emission may provide the user with a visual indication that the power source requires recharging.

The predetermined threshold level of energy is preferably less than or equal to 20% of a predetermined energy capacity of the power source.

The control electronics may be configured to activate either or both of the first and second lighting arrays such that the low energy light emission has a predetermined colour.

The control electronics may be configured to activate a minor proportion of either or both of the first and second lighting arrays to generate the low energy light emission. Preferably, the minor proportion forms less than 15%, or preferably less than 10%, or preferably less than 5% of the length of either or both of the first and second lighting arrays. The minor proportion may be located at one of the first or second ends of either or both of the first and second lighting arrays.

Conveniently, the aerosol-generating device may further comprise: a power source coupled to the control electronics. The control electronics may be configured to: receive a selection input selecting one of at least a first and a second predetermined thermal profile. Each of the first and second predetermined thermal profiles may define a heating profile for heating of the aerosol-forming substrate by an electrical heating arrangement over the usage session. The first and second predetermined thermal profiles are different to each other. The control electronics may also be configured to: control a supply of energy from the power source to the electrical heating arrangement to heat the aerosol-forming substrate in accordance with the selected thermal profile; and activate either or both of the first and second lighting arrays to generate a first thermal profile light emission in response to selection of the first predetermined thermal profile and activate either or both of the first and second lighting arrays to generate a second thermal profile light emission in response to selection of the second predetermined thermal profile. The first thermal profile light emission is indicative of the selection of the first predetermined thermal profile. The second thermal profile light emission is indicative of the selection of the second predetermined thermal profile. In this manner, a user may be provided with a visual indication as to which of the predetermined thermal profiles has been selected for heating of the aerosol-forming substrate.

The second predetermined thermal profile may have a greater intensity than the first predetermined thermal profile. Conveniently, the second predetermined thermal profile is associated with supply of a greater amount of energy from the power source to the electrical heating arrangement over the usage session than for the first predetermined thermal profile.

The aerosol-generating device may comprise a user interface actuatable by a user to select between the first and second predetermined thermal profiles. Preferably, the user interface comprises a button, or a motion sensor.

The control electronics may be configured to generate the selection input in response to the user selecting between the first and second predetermined thermal profiles via the user interface.

The control electronics may be configured to: activate a first proportion of either or both of the first and second lighting arrays to generate the first thermal profile light emission in response to selection of the first predetermined thermal profile; and activate a second proportion of either or both of the first and second lighting arrays to generate the second thermal profile light emission in response to selection of the second predetermined thermal profile. The second proportion may be greater than the first proportion. Preferably, the second proportion defines a greater proportion of a combined length of the first and second lighting arrays than the first proportion.

The first and second lighting arrays may collectively comprise a plurality of lighting elements. The control electronics may also be configured to activate a greater number of the plurality of lighting elements to generate the second thermal profile light emission than to generate the first thermal profile light emission.

The control electronics may be configured to: activate all or part of only the first lighting array to generate the first thermal profile light emission; and activate all or part of only the second lighting array to generate the second thermal profile light emission. Preferably, the control electronics may be configured to: activate a first percentage of the length of the first lighting array to generate the first thermal profile light emission; and activate a second percentage of the length of the second lighting array to generate the second thermal profile light emission. The second percentage value may be greater than the first percentage value.

The control electronics may be configured to activate either or both of the first and second lighting arrays such that the first thermal profile light emission and the second thermal profile light emission differ from each other in one or more of luminance and colour. Preferably, the control electronics may be configured to activate either or both of the first and second lighting arrays such that the first thermal profile light emission has a first predetermined colour and the second thermal profile light emission has a second predetermined colour. A dominant wavelength of the second thermal profile light emission may be greater in size than a dominant wavelength of the first thermal profile light emission.

Conveniently, the aerosol-generating device may further comprise: a power source coupled to the control electronics. The control electronics may be configured to: control a supply of energy from the power source to an electrical heating arrangement to heat the aerosol-forming substrate at a first temperature level in an aerosol-releasing mode; in response to a pause signal, control the supply of energy from the power source to the electrical heating arrangement to heat the aerosol-forming substrate at a second temperature level below the first temperature level in a pause mode; and activate either or both of the first and second lighting arrays to generate a pause light emission in response to the pause signal. The pause light emission is indicative of the aerosol-generating device being in the pause mode. In this manner, a user may be provided with a visual indication of the aerosol-generating device being in the pause mode.

The aerosol-generating device may comprise a motion sensor for detecting a movement of the aerosol-generating device, the motion sensor coupled to the control electronics. The control electronics may be configured to use the detected movement to trigger the pause signal.

The aerosol-generating device may comprise a motion sensor for detecting a lack of movement of the aerosol-generating device, the motion sensor coupled to the control electronics. The control electronics may be configured to use the lack of detected movement to trigger the pause signal. Preferably, the lack of movement of the aerosol-generating device is detected by an absence of movement of the device for a predetermined amount of time, or an absence of movement above a certain magnitude for a predetermined amount of time.

The aerosol-generating device may further comprise a user interface and/or a puff detection mechanism for detecting puffs on the device. The control electronics may be configured to trigger the pause signal in response to detecting an absence of a user interaction with the user interface and/or with the puff detection mechanism for a predetermined amount of time. Preferably, the control electronics may be configured to use the detected movement to trigger the pause signal when the detected movement corresponds to a predetermined movement.

The aerosol-generating device may comprise an orientation sensor for detecting an orientation of the aerosol-generating device, the orientation sensor coupled to the control electronics. The control electronics may be configured to use the detected orientation, or an absence of a change in the detected orientation for a predetermined length of time, to trigger the pause signal. Preferably, the control electronics may be configured to use the detected orientation to trigger the pause signal when the detected orientation corresponds to a predetermined orientation.

The aerosol-generating device may further comprise a user interface actuatable by a user to initiate the pause mode, preferably wherein the user interface comprises a button. Preferably, the control electronics may be configured to generate the pause signal in response to the user initiating the pause mode via the user interface, or in response to detecting an absence of a user interacting with the user interface after a predetermined length of time.

The control electronics may be configured to activate a portion of each of the first and second lighting arrays to generate the pause light emission. The first and second lighting arrays may be arranged relative to each other such that the respective portions of the first and second lighting arrays are parallel with each other. The respective portions of the first and second lighting arrays may each have substantially the same length. Preferably, the control electronics may be configured to sequentially activate and deactivate the respective portions of the first and second lighting arrays to generate the pause light emission. The control electronics may be configured to activate and deactivate the respective portions of the first and second lighting arrays out of phase with each other to generate the pause light emission.

The control electronics may be configured to activate the respective portions of the first and second lighting arrays to change with time in at least one of luminance or wavelength, so as to vary the luminance or colour of the pause light emission with respect to time.

The control electronics may be configured to activate all or part of each of the first and second lighting arrays to generate the pause light emission such that a centre portion of each of the first and second lighting arrays has a luminance greater than the remainder of the respective lighting array. Preferably, the control electronics may be configured to activate all or part of each of the first and second lighting arrays to generate the pause light emission such that the luminance of each of the first and second lighting arrays progressively decreases when moving from the centre portion towards the first and second ends of the respective lighting array.

Conveniently, the aerosol-generating device may further comprise: a power source coupled to the control electronics. The control electronics may be configured to: receive an input to change an operational state of the aerosol-generating device; control a supply of energy from the power source to change the operational state; and activate either or both of the first and second lighting arrays to generate a state change light emission in response to the input. The state change light emission may be indicative of the receiving of the input to change the operational state. In this manner, a user may be provided with a visual indication of a change in the operational state of the aerosol-generating device.

The change of operational state may comprise activation of the device from an off mode, or reactivation of the device from a pause mode. Preferably, the reactivation of the device may correspond to a supply of energy from the power source to an electrical heating arrangement being so as to heat the aerosol-forming substrate at a first temperature level in an aerosol-releasing mode. Further, the pause mode may correspond to the supply of energy from the power source to the electrical heating arrangement being so as to heat the aerosol-forming substrate at a second temperature level below the first temperature level.

The control electronics may be configured to progressively activate each of the first and second lighting arrays over a predetermined time period so as to progressively increase an activated length of each of the first and second lighting arrays over the predetermined time period for the state change light emission.

The control electronics may be configured to activate all or part of each of the first and second lighting arrays to progressively increase in luminance over a predetermined time period for the state change light emission. Preferably, the control electronics may be configured to activate all or part of each of the first and second lighting arrays such that at the start of the predetermined time period the luminance of the activated part of each of the first and second lighting arrays progressively reduces with distance away from a centre of the activated part towards the first and second ends of the respective lighting array. The luminance may progressively increase over the predetermined time period such that at the end of the predetermined time period the activated part of each of the first and second lighting arrays has a uniform luminance over the length of the activated part of the respective lighting array.

The control electronics may be configured to activate all or part of each of the first and second lighting arrays such that the luminance of the activated part of each of the first and second predetermined lighting arrays is symmetric about a centre of the activated part of the respective lighting array over the predetermined time period.

Conveniently, the aerosol-generating device may further comprise: a power source coupled to the control electronics. The control electronics may be configured to: control a supply of energy from the power source to an electrical heating arrangement over the usage session to heat the aerosol-forming substrate; determine progression through the usage session by reference to a parameter indicative of progression through the usage session; and activate either or both of the first and second lighting arrays to generate a progressing light emission which varies according to progression through the usage session, such that the progressing light emission is indicative of progression through the usage session. In this manner, a user may be provided with a visual indication of progression through the usage session.

The parameter indicative of progression through the usage session may comprise one or more of: a cumulative time elapsed since the start of the usage session, a cumulative puff count of a series of puffs drawn by a user since the start of the usage session, and a cumulative volume of aerosol evolved from the aerosol-forming substrate since the start of the usage session.

The control electronics may be configured to reduce or terminate the supply of energy from the power source to the electrical heating arrangement to complete the usage session upon the occurrence of the cumulative time elapsed since the start of the usage session reaching a predetermined maximum time duration.

The control electronics may be configured to reduce or terminate the supply of energy from the power source to the electrical heating arrangement to complete the usage session upon the first to occur of: i) the cumulative time elapsed since the start of the usage session reaching the predetermined maximum time duration; and ii) the cumulative puff count reaching a predetermined maximum number of puffs.

The control electronics may be configured to reduce or terminate the supply of energy from the power source to the electrical heating arrangement to complete the usage session upon the first to occur of: i) the cumulative time elapsed since the start of the usage session reaching the predetermined maximum time duration; and ii) the cumulative volume of aerosol reaching a predetermined volume limit.

The control electronics may be configured to activate all or a major portion of the first lighting array at the start of a first usage session; and to progressively deactivate the first lighting array so as to progressively reduce an activated length of the first lighting array with progression through the first usage session. The control electronics may further be configured to activate all or a major portion of the second lighting array at the start of a second usage session; and to progressively deactivate the second lighting array so as to progressively reduce an activated length of the second lighting array with progression through the second usage session. Conveniently, the control electronics may be configured such that: on completion of the first usage session, no light is emitted from the first lighting array; and on completion of the second usage session, no light is emitted from the second lighting array.

The control electronics may be configured to: maintain the second lighting array in a deactivated state over the first usage session; and maintain the first lighting array in a deactivated state over the second usage session.

The first and second lighting arrays may be arranged parallel to each other. The control electronics may be configured to activate all or a major portion of both the first lighting array and the second lighting array at the start of the usage session. The control electronics may further be configured to progressively deactivate the first and second lighting arrays in synchronization with each other so as to progressively reduce an activated length of each of the first and second lighting arrays with progression through the usage session, such that the respective activated lengths of the first and second lighting arrays remain equal during the usage session.

The first and second lighting arrays may be arranged parallel to each other in a collective arrangement, in which the collective arrangement of the first and second lighting arrays has a length and a width. The control electronics may be configured to activate all or a major portion of both the first lighting array and the second lighting array at the start of the usage session. The control electronics may further be configured to progressively deactivate the first and second lighting arrays in a snakewise manner across the width and along the length of the collective arrangement so as to progressively reduce an activated length of each of the first and second lighting arrays with progression through the usage session.

The aerosol-generating device may further comprise a substantially linear third lighting array located between and parallel to each of the first and second lighting arrays. The control electronics may be configured to activate all or a major portion of the third lighting array at the start of the usage session. The control electronics may further be configured to progressively deactivate the third lighting array so as to progressively reduce an activated length of the third lighting array with progression through the usage session. Preferably, the control electronics may be configured such that on completion of the usage session, no light is emitted from the third lighting array. Preferably, the control electronics may be configured to maintain the first and second lighting arrays in a deactivated state over the usage session.

Conveniently, the aerosol-generating device may further comprise: a power source coupled to the control electronics. The aerosol-generating device may be configured to receive an aerosol-generating article comprising the aerosol-forming substrate. The control electronics may be configured to: detect the receiving of the aerosol-generating article by the aerosol-generating device; control a supply of energy from the power source to an electrical heating arrangement to activate a pre-heating phase in which the electrical heating arrangement is heated to a predetermined target temperature; and activate either or both of the first and second lighting arrays to generate a pre-heating light emission. The pre-heating light emission may vary according to progression through the pre-heating phase so as to be indicative of progression through the pre-heating phase. In this manner, a user may be provided with a visual indication of progression through the pre-heating phase.

The aerosol-generating device may comprise a cavity for receiving the aerosol-generating article. The aerosol-generating article may comprise an inductively heatable susceptor. The electrical heating arrangement may comprise an inductive heating arrangement coupled to the power source and configured to generate an alternating magnetic field within the cavity for inductively heating the susceptor of the aerosol-generating article when the aerosol-generating article is received in the cavity. The control circuitry may be configured to: generate probe power pulses for intermittently powering on the inductive heating arrangement; and detect a change of at least one property of the inductive heating arrangement due to the presence of the susceptor when an aerosol-generating article is received in the cavity, thereby enabling detection of the aerosol-generating article being received in the cavity. Preferably, the at least one property may be an equivalent resistance of the inductive heating arrangement or an inductance of the inductive heating arrangement.

The control electronics may be configured to activate the first and second lighting arrays so that different portions of each of the first and second lighting arrays vary in luminance with time and with respect to each other, and the luminance of each of the first and second lighting arrays progressively increases over the pre-heating phase.

The control electronics may be configured to activate the first and second lighting arrays such that a dominant wavelength of the predetermined emission of light progressively increases over the pre-heating phase.

The first and second lighting arrays may be arranged parallel to each other. The control electronics may be configured to: activate the first and second lighting arrays in synchronization with each other so as to progressively increase an activated length of each of the first and second lighting arrays with progression through the pre-heating phase, such that the respective activated lengths of the first and second lighting arrays remain equal during the usage session.

The first and second lighting arrays may be arranged parallel to each other in a collective arrangement, in which the collective arrangement has a length and a width. The control electronics may be configured to: activate the first and second lighting arrays in a snakewise manner across the width and along the length of the collective arrangement so as to progressively increase an activated length of each of the first and second lighting arrays with progression through the pre-heating phase.

The control electronics may be configured to activate the first and second lighting arrays such that during or upon completion of the pre-heating phase the luminance of respective activated lengths of the first and second lighting arrays progressively increase with distance between first and second opposed ends of the respective activated length.

The control electronics may be configured to activate the first and second lighting arrays such that during or upon completion of the pre-heating phase a dominant wavelength of the pre-heating light emission progressively increases with distance between first and second opposed ends of respective activated lengths of the first and second lighting arrays. Preferably, the dominant wavelength may be within a range of 380 to 750 nanometres, such that during or upon completion of the pre-heating phase the first opposed end defines a blue colour for the pre-heating light emission and the second opposed end defines a red colour for the pre-heating light emission.

The control electronics may be configured such that both of the first and second lighting arrays have a uniform luminance along the length of the respective lighting array upon completion of the pre-heating phase.

Preferably, the aerosol-forming substrate is a solid aerosol-forming substrate. However, the aerosol-forming substrate may comprise both solid and liquid components. Alternatively, the aerosol-forming substrate may be a liquid aerosol-forming substrate.

Preferably, the aerosol-forming substrate comprises nicotine. More preferably, the aerosol-forming substrate comprises tobacco. Alternatively or in addition, the aerosol-forming substrate may comprise a non-tobacco containing aerosol-forming material.

If the aerosol-forming substrate is a solid aerosol-forming substrate, the solid aerosol-forming substrate may comprise, for example, one or more of: powder, granules, pellets, shreds, strands, strips or sheets containing one or more of: herb leaf, tobacco leaf, tobacco ribs, expanded tobacco and homogenised tobacco.

Optionally, the solid aerosol-forming substrate may contain tobacco or non-tobacco volatile flavour compounds, which are released upon heating of the solid aerosol-forming substrate. The solid aerosol-forming substrate may also contain one or more capsules that, for example, include additional tobacco volatile flavour compounds or non-tobacco volatile flavour compounds and such capsules may melt during heating of the solid aerosol-forming substrate.

Optionally, the solid aerosol-forming substrate may be provided on or embedded in a thermally stable carrier. The carrier may take the form of powder, granules, pellets, shreds, strands, strips or sheets. The solid aerosol-forming substrate may be deposited on the surface of the carrier in the form of, for example, a sheet, foam, gel or slurry. The solid aerosol-forming substrate may be deposited on the entire surface of the carrier, or alternatively, may be deposited in a pattern in order to provide a non-uniform flavour delivery during use.

In a preferred embodiment, the aerosol-forming substrate comprises homogenised tobacco material. As used herein, the term “homogenised tobacco material” refers to a material formed by agglomerating particulate tobacco.

Preferably, the aerosol-forming substrate comprises a gathered sheet of homogenised tobacco material. As used herein, the term “sheet” refers to a laminar element having a width and length substantially greater than the thickness thereof. As used herein, the term “gathered” is used to describe a sheet that is convoluted, folded, or otherwise compressed or constricted substantially transversely to the longitudinal axis of the aerosol-generating article.

Preferably, the aerosol-forming substrate comprises an aerosol former. As used herein, the term “aerosol former” is used to describe any suitable known compound or mixture of compounds that, in use, facilitates formation of an aerosol and that is substantially resistant to thermal degradation at the operating temperature of the aerosol-generating article.

Suitable aerosol-formers are known in the art and include, but are not limited to: polyhydric alcohols, such as propylene glycol, triethylene glycol, 1,3-butanediol and glycerine; esters of polyhydric alcohols, such as glycerol mono-, di- or triacetate; and aliphatic esters of mono-, di- or polycarboxylic acids, such as dimethyl dodecanedioate and dimethyl tetradecanedioate. Preferred aerosol formers are polyhydric alcohols or mixtures thereof, such as propylene glycol, triethylene glycol, 1,3-butanediol and, most preferred, glycerine.

The aerosol-forming substrate may comprise a single aerosol former. Alternatively, the aerosol-forming substrate may comprise a combination of two or more aerosol formers.

The invention is defined in the claims. However, below there is provided a non-exhaustive list of non-limiting examples. Any one or more of the features of these examples may be combined with any one or more features of another example, embodiment, or aspect described herein.

Example Ex1: An aerosol-generating device for heating an aerosol-forming substrate to generate an inhalable aerosol during a usage session, the aerosol-generating device comprising: control electronics; a substantially linear first lighting array and a substantially linear second lighting array, each of the first and second lighting arrays extending over a length between first and second ends of the respective lighting array; in which the control electronics are coupled to the first and second lighting arrays and configured to activate either or both of the first and second lighting arrays to generate a predetermined light emission indicative of and in response to at least one of: i) a status of the aerosol-generating device; and ii) progression of an operational phase of the aerosol-generating device.

Example Ex2: An aerosol-generating device according to Ex1, in which the control electronics are configured to activate either or both of the first and second lighting arrays at two or more luminance levels, so as to control the luminance of the predetermined light emission.

Example Ex3: An aerosol-generating device according to either one of Ex1 or Ex2, in which the control electronics are configured to activate either or both of the first and second lighting arrays in two or more colour states, so as to control the colour of the predetermined light emission.

Example Ex4: An aerosol-generating device according to any one of Ex1 to Ex3, in which the control electronics are configured to activate either or both of the first and second lighting arrays so as to vary the predetermined light emission with respect to time.

Example Ex5: An aerosol-generating device according to Ex4, in which the control electronics are configured to activate either or both of first and second lighting arrays to vary the predetermined light emission with respect to time so as to be indicative of the progression of the operational phase of the aerosol-generating device.

Example Ex6: An aerosol-generating device according to Ex5, wherein the progression of the operational phase is the progression of the usage session.

Example Ex7: An aerosol-generating device according to any one of Ex4 to Ex6, in which the control electronics are configured to activate either or both of the first and second lighting arrays so as to vary an activated length of the respective lighting array with respect to time.

Example Ex8: An aerosol-generating device according to any one of Ex4 to Ex7, in which the control electronics are configured to vary the predetermined light emission with respect to time in one or more of luminance and colour.

Example Ex9: An aerosol-generating device according to any one of Ex4 to Ex8, in which the control electronics are configured to vary the predetermined lighting emission with respect to time by one or more of activating, deactivating and reactivating different portions of either or both of the first and second lighting arrays over time.

Example Ex10: An aerosol-generating device according to any one of Ex1 to Ex9, in which each of the first and second lighting arrays comprise a plurality of light emitting units distributed between the first and second ends of the respective lighting array.

Example Ex11: An aerosol-generating device according to Ex10, further comprising one or more waveguides configured to direct light generated by one or more of the plurality of light emitting units to one or more display windows for viewing of the predetermined light emission by a user.

Example Ex12: An aerosol-generating device according to either one of Ex10 or Ex11, wherein each one of the plurality of light emitting units comprises a light emitting diode and the control electronics comprises a light emitting diode control driver and a separate microcontroller, the control driver configured to control a supply of electricity from a power source to one or more of the plurality of light emitting diodes of either or both of the first and second lighting arrays under the control of the microcontroller, so as to generate the predetermined light emission.

Example Ex13: An aerosol-generating device according to Ex12, in which the plurality of light emitting diodes of each of the first and second lighting arrays comprises: a first set of one or more light emitting diodes configured to emit light of a first colour; and a second set of one or more light emitting diodes configured to emit light of a second colour; in which the light emitting diode control driver is configured to activate one or more of the light emitting diodes from the first set alone of either or both of the first and second lighting arrays, or from the second set alone of either or both of the first and second lighting arrays, or from both of the first and second sets of either or both of the first and second lighting arrays, so as to control the colour of the predetermined light emission.

Example Ex14: An aerosol-generating device according to either one of Ex12 or Ex13, in which the light emitting diode control driver is configured to control a supply of electricity from a power source to one or more of the plurality of light emitting diodes of either or both of the first and second lighting arrays by a pulse width modulation regime having a predetermined resolution, so as to control the luminance of the predetermined light emission, in which the predetermined resolution defines two or more luminance levels.

Example Ex15: An aerosol-generating device according to any one of Ex1 to Ex14, in which the length of the first lighting array is the same as the length of the second lighting array.

Example Ex16: An aerosol-generating device according to any one of Ex1 to Ex15, in which the first lighting array and the second lighting array are laterally spaced apart from each other and parallel to each other.

Example Ex17: An aerosol-generating device according to any one of Ex1 to Ex16, in which the predetermined light emission is one or more of a usage session light emission, a low energy light emission, a thermal profile light emission, a pause light emission, a state change light emission, a progressing light emission and a pre-heating light emission.

Example Ex18: An aerosol-generating device according to any one of Ex1 to Ex17, further comprising a substantially linear third lighting array located between and parallel to each of the first and second lighting arrays, in which the control electronics are configured to activate the third lighting array alone or in addition to either or both of the first and second lighting array to generate the predetermined light emission.

Example Ex19: An aerosol-generating device according to any one of Ex1 to Ex18, the aerosol-generating device further comprising: a power source coupled to the control electronics; in which the control electronics are configured to: determine a level of energy contained in the power source; compare the determined level of energy with first and second predetermined energy thresholds, the first predetermined energy threshold corresponding to the power source containing sufficient energy to complete a single usage session, the second predetermined energy threshold corresponding to the power source containing sufficient energy to complete two or more usage sessions; activate either or both of the first and second lighting arrays to generate a single usage session light emission in response to a first state in which the determined level of energy is sufficient to complete a single usage session; activate either or both of the first and second lighting arrays to generate a plural usage sessions light emission in response to a second state in which the determined level of energy is sufficient to complete two or more usage sessions; wherein the single usage session light emission and the plural usage sessions light emission are different to each other, in which the single usage session light emission is indicative of the first state and the plural usage sessions light emission is indicative of the second state.

Example Ex20: An aerosol-generating device according to Ex19, in which the control electronics are configured to activate a greater proportion of either or both of the first and second lighting arrays to generate the plural usage sessions light emission than to generate the single usage session light emission.

Example Ex21: An aerosol-generating device according to either one of Ex19 or Ex20, in which the control electronics are configured to: activate all or part of only the first lighting array to generate the single usage session light emission in response to the first state; and activate all or part of both the first lighting array and the second lighting array to generate the plural usage sessions light emission in response to the second state.

Example Ex22: An aerosol-generating device according Ex21, in which the control electronics are configured to: activate between 90% to 100% of the length of the first lighting array to generate the single usage session light emission; and activate between 90% to 100% of the length of both the first lighting array and the second lighting array to generate the plural usage sessions light emission.

Example Ex23: An aerosol-generating device according to any one of Ex19 to Ex22, in which the control electronics are configured to activate either or both of the first and second lighting arrays such that the single usage session light emission and the plural usage sessions light emission are different to each other in one or more of luminance and colour.

Example Ex24: An aerosol-generating device according to Ex23, in which the control electronics are configured to activate either or both of the first and second lighting arrays such that the single usage session light emission has a first predetermined luminance and the plural usage sessions light emission has a second predetermined luminance, the second predetermined luminance being greater than the first predetermined luminance.

Example Ex25: An aerosol-generating device according to any one of Ex1 to Ex24, the aerosol-generating device further comprising: a power source coupled to the control electronics; in which the control electronics are configured to: determine a level of energy contained in the power source and compare the determined level of energy with a predetermined threshold level of energy; and activate either or both of the first and second lighting arrays to generate a low energy light emission in response to the determined level of energy being less than or equal to the predetermined threshold level of energy, in which the low energy light emission is indicative of the determined level of energy being less than or equal to the predetermined threshold level of energy.

Example Ex26: An aerosol-generating device according to Ex25, in which the predetermined threshold level of energy is less than or equal to 20% of a predetermined energy capacity of the power source.

Example Ex27: An aerosol-generating device according to either one of Ex25 or Ex26, in which the control electronics are configured to activate either or both of the first and second lighting arrays such that the low energy light emission has a predetermined colour.

Example Ex28: An aerosol-generating device according to any one of Ex25 to Ex27, in which the control electronics are configured to activate a minor proportion of either or both of the first and second lighting arrays to generate the low energy light emission.

Example Ex29: An aerosol-generating device according to Ex28, in which the minor proportion forms less than 15%, or preferably less than 10%, or preferably less than 5% of the length of either or both of the first and second lighting arrays.

Example Ex30: An aerosol-generating device according to either one of Ex28 or Ex29, in which the minor proportion is located at one of the first or second ends of either or both of the first and second lighting arrays.

Example Ex31: An aerosol-generating device according to any one of Ex1 to Ex30, the aerosol-generating device further comprising: a power source coupled to the control electronics; in which the control electronics are configured to: receive a selection input selecting one of at least a first and a second predetermined thermal profile, in which each of the first and second predetermined thermal profiles define a heating profile for heating of the aerosol-forming substrate by an electrical heating arrangement over the usage session, the first and second predetermined thermal profiles being different to each other; control a supply of energy from the power source to the electrical heating arrangement to heat the aerosol-forming substrate in accordance with the selected thermal profile; activate either or both of the first and second lighting arrays to generate a first thermal profile light emission in response to selection of the first predetermined thermal profile and activate either or both of the first and second lighting arrays to generate a second thermal profile light emission in response to selection of the second predetermined thermal profile, in which the first thermal profile light emission is indicative of the selection of the first predetermined thermal profile and the second thermal profile light emission is indicative of the selection of the second predetermined thermal profile.

Example Ex32: An aerosol-generating device according to Ex31, in which the second predetermined thermal profile has a greater intensity than the first predetermined thermal profile.

Example Ex33: An aerosol-generating device according to Ex32, in which the second predetermined thermal profile is associated with supply of a greater amount of energy from the power source to the electrical heating arrangement over the usage session than for the first predetermined thermal profile.

Example Ex34: An aerosol-generating device according to any one of Ex31 to Ex33, in which the aerosol-generating device comprises a user interface actuatable by a user to select between the first and second predetermined thermal profiles, preferably wherein the user interface comprises a button, or a motion sensor.

Example Ex35: An aerosol-generating device according to Ex34, in which the control electronics are configured to generate the selection input in response to the user selecting between the first and second predetermined thermal profiles via the user interface.

Example Ex36: An aerosol-generating device according to any one of Ex31 to Ex35, in which the control electronics are configured to: activate a first proportion of either or both of the first and second lighting arrays to generate the first thermal profile light emission in response to selection of the first predetermined thermal profile; and activate a second proportion of either or both of the first and second lighting arrays to generate the second thermal profile light emission in response to selection of the second predetermined thermal profile; the second proportion being greater than the first proportion.

Example Ex37: An aerosol-generating device according to Ex36, in which the second proportion defines a greater proportion of a combined length of the first and second lighting arrays than the first proportion.

Example Ex38: An aerosol-generating device according to any one of Ex31 to Ex37, in which the first and second lighting arrays collectively comprise a plurality of lighting elements, in which the control electronics are configured to activate a greater number of the plurality of lighting elements to generate the second thermal profile light emission than to generate the first thermal profile light emission.

Example Ex39: An aerosol-generating device according to any one of Ex31 to Ex38, in which the control electronics are configured to: activate all or part of only the first lighting array to generate the first thermal profile light emission; and activate all or part of only the second lighting array to generate the second thermal profile light emission.

Example Ex40: An aerosol-generating device according to Ex39, in which the control electronics are configured to: activate a first percentage of the length of the first lighting array to generate the first thermal profile light emission; and activate a second percentage of the length of the second lighting array to generate the second thermal profile light emission; in which the second percentage value is greater than the first percentage value.

Example Ex41: An aerosol-generating device according to any one of Ex31 to Ex40, in which the control electronics are configured to activate either or both of the first and second lighting arrays such that the first thermal profile light emission and the second thermal profile light emission differ from each other in one or more of luminance and colour.

Example Ex42: An aerosol-generating device according to Ex41, in which the control electronics are configured to activate either or both of the first and second lighting arrays such that the first thermal profile light emission has a first predetermined colour and the second thermal profile light emission has a second predetermined colour, in which a dominant wavelength of the second thermal profile light emission is greater in size than a dominant wavelength of the first thermal profile light emission.

Example Ex43: An aerosol-generating device according to any one of Ex1 to Ex42, the aerosol-generating device further comprising: a power source coupled to the control electronics; in which the control electronics are configured to: control a supply of energy from the power source to an electrical heating arrangement to heat the aerosol-forming substrate at a first temperature level in an aerosol-releasing mode; in response to a pause signal, control the supply of energy from the power source to the electrical heating arrangement to heat the aerosol-forming substrate at a second temperature level below the first temperature level in a pause mode; and activate either or both of the first and second lighting arrays to generate a pause light emission in response to the pause signal, in which the pause light emission is indicative of the aerosol-generating device being in the pause mode.

Example Ex44: An aerosol-generating device according to Ex43, in which the aerosol-generating device comprises a motion sensor for detecting a movement of the aerosol-generating device, the motion sensor coupled to the control electronics, the control electronics configured to use the detected movement to trigger the pause signal.

Example Ex45: An aerosol-generating device according to Ex44, in which the aerosol-generating device comprises a motion sensor for detecting a lack of movement of the aerosol-generating device, the motion sensor coupled to the control electronics, the control electronics configured to use the lack of detected movement to trigger the pause signal.

Example Ex46: An aerosol-generating device according to Ex45, wherein the lack of movement of the aerosol-generating device is detected by an absence of movement of the device for a predetermined amount of time, or an absence of movement above a certain magnitude for a predetermined amount of time.

Example Ex47: An aerosol-generating device according to Ex43, further comprising a user interface and/or a puff detection mechanism for detecting puffs on the device, wherein the control electronics are configured to trigger the pause signal in response to detecting an absence of a user interaction with the user interface and/or with the puff detection mechanism for a predetermined amount of time.

Example Ex48: An aerosol-generating device according to Ex44, in which the control electronics are configured to use the detected movement to trigger the pause signal when the detected movement corresponds to a predetermined movement.

Example Ex49: An aerosol-generating device according to any one of Ex43 to Ex48, in which the aerosol-generating device comprises an orientation sensor for detecting an orientation of the aerosol-generating device, the orientation sensor coupled to the control electronics, the control electronics configured to use the detected orientation, or an absence of a change in the detected orientation for a predetermined length of time, to trigger the pause signal.

Example Ex50: An aerosol-generating device according to Ex49, in which the control electronics are configured to use the detected orientation to trigger the pause signal when the detected orientation corresponds to a predetermined orientation.

Example Ex51: An aerosol-generating device according to any one of Ex43 to Ex50, in which the aerosol-generating device further comprises a user interface actuatable by a user to initiate the pause mode, preferably wherein the user interface comprises a button.

Example Ex52: An aerosol-generating device according to Ex51, in which the control electronics are configured to generate the pause signal in response to the user initiating the pause mode via the user interface, or in response to detecting an absence of a user interacting with the user interface after a predetermined length of time.

Example Ex53: An aerosol-generating device according to any one of Ex43 to Ex52, in which the control electronics are configured to activate a portion of each of the first and second lighting arrays to generate the pause light emission, in which the first and second lighting arrays are arranged relative to each other such that the respective portions of the first and second lighting arrays are parallel with each other, the respective portions of the first and second lighting arrays having substantially the same length.

Example Ex54: An aerosol-generating device according to Ex53, in which the control electronics are configured to sequentially activate and deactivate the respective portions of the first and second lighting arrays to generate the pause light emission.

Example Ex55: An aerosol-generating device according to Ex54, in which the control electronics are configured to activate and deactivate the respective portions of the first and second lighting arrays out of phase with each other to generate the pause light emission.

Example Ex56: An aerosol-generating device according to any one of Ex53 to Ex55, in which the control electronics are configured to activate the respective portions of the first and second lighting arrays to change with time in at least one of luminance or wavelength, so as to vary the luminance or colour of the pause light emission with respect to time.

Example Ex57: An aerosol-generating device according to any one of Ex43 to Ex56, in which the control electronics are configured to activate all or part of each of the first and second lighting arrays to generate the pause light emission such that a centre portion of each of the first and second lighting arrays has a luminance greater than the remainder of the respective lighting array.

Example Ex58: An aerosol-generating device according to Ex57, in which the control electronics are configured to activate all or part of each of the first and second lighting arrays to generate the pause light emission such that the luminance of each of the first and second lighting arrays progressively decreases when moving from the centre portion towards the first and second ends of the respective lighting array.

Example Ex59: An aerosol-generating device according to any one of Ex1 to Ex58, the aerosol-generating device further comprising: a power source coupled to the control electronics; in which the control electronics are configured to: receive an input to change an operational state of the aerosol-generating device; control a supply of energy from the power source to change the operational state; and activate either or both of the first and second lighting arrays to generate a state change light emission in response to the input, in which the state change light emission is indicative of the receiving of the input to change the operational state.

Example Ex60: An aerosol-generating device according to Ex59, in which the change of operational state comprises activation of the device from an off mode, or reactivation of the device from a pause mode.

Example Ex61: An aerosol-generating device according to Ex60, in which the reactivation of the device corresponds to a supply of energy from the power source to an electrical heating arrangement being so as to heat the aerosol-forming substrate at a first temperature level in an aerosol-releasing mode, and in which the pause mode corresponds to the supply of energy from the power source to the electrical heating arrangement being so as to heat the aerosol-forming substrate at a second temperature level below the first temperature level.

Example Ex62: An aerosol-generating device according to any one of Ex59 to Ex61, in which the control electronics are configured to progressively activate each of the first and second lighting arrays over a predetermined time period so as to progressively increase an activated length of each of the first and second lighting arrays over the predetermined time period for the state change light emission.

Example Ex63: An aerosol-generating device according to any one of Ex59 to Ex62, in which the control electronics are configured to activate all or part of each of the first and second lighting arrays to progressively increase in luminance over a predetermined time period for the state change light emission.

Example Ex64: An aerosol-generating device according to Ex63, in which the control electronics are configured to activate all or part of each of the first and second lighting arrays such that at the start of the predetermined time period the luminance of the activated part of each of the first and second lighting arrays progressively reduces with distance away from a centre of the activated part towards the first and second ends of the respective lighting array, with the luminance progressively increasing over the predetermined time period such that at the end of the predetermined time period the activated part of each of the first and second lighting arrays has a uniform luminance over the length of the activated part of the respective lighting array.

Example Ex65: An aerosol-generating device according to either one of Ex63 or Ex64, in which the control electronics are configured to activate all or part of each of the first and second lighting arrays such that the luminance of the activated part of each of the first and second predetermined lighting arrays is symmetric about a centre of the activated part of the respective lighting array over the predetermined time period.

Example Ex66: An aerosol-generating device according to any one of Ex1 to Ex65, the aerosol-generating device further comprising: a power source coupled to the control electronics; in which the control electronics are configured to: control a supply of energy from the power source to an electrical heating arrangement over the usage session to heat the aerosol-forming substrate; determine progression through the usage session by reference to a parameter indicative of progression through the usage session; and activate either or both of the first and second lighting arrays to generate a progressing light emission which varies according to progression through the usage session, such that the progressing light emission is indicative of progression through the usage session.

Example Ex67: An aerosol-generating device according to Ex66, in which the parameter indicative of progression through the usage session comprises one or more of: a cumulative time elapsed since the start of the usage session, a cumulative puff count of a series of puffs drawn by a user since the start of the usage session, and a cumulative volume of aerosol evolved from the aerosol-forming substrate since the start of the usage session.

Example Ex68: An aerosol-generating device according to Ex67, in which the control electronics are configured to reduce or terminate the supply of energy from the power source to the electrical heating arrangement to complete the usage session upon the occurrence of the cumulative time elapsed since the start of the usage session reaching a predetermined maximum time duration.

Example Ex69: An aerosol-generating device according to Ex68, in which the control electronics are configured to reduce or terminate the supply of energy from the power source to the electrical heating arrangement to complete the usage session upon the first to occur of: i) the cumulative time elapsed since the start of the usage session reaching the predetermined maximum time duration; and ii) the cumulative puff count reaching a predetermined maximum number of puffs.

Example Ex70: An aerosol-generating device according to either one of Ex68 or Ex69, in which the control electronics are configured to reduce or terminate the supply of energy from the power source to the electrical heating arrangement to complete the usage session upon the first to occur of: i) the cumulative time elapsed since the start of the usage session reaching the predetermined maximum time duration; and ii) the cumulative volume of aerosol reaching a predetermined volume limit.

Example Ex71: An aerosol-generating device according to any one of Ex66 to Ex70, in which the control electronics are configured to activate all or a major portion of the first lighting array at the start of a first usage session; progressively deactivate the first lighting array so as to progressively reduce an activated length of the first lighting array with progression through the first usage session; activate all or a major portion of the second lighting array at the start of a second usage session; progressively deactivate the second lighting array so as to progressively reduce an activated length of the second lighting array with progression through the second usage session.

Example Ex72: An aerosol-generating device according to Ex71, in which the control electronics are configured such that: on completion of the first usage session, no light is emitted from the first lighting array; and on completion of the second usage session, no light is emitted from the second lighting array.

Example Ex73: An aerosol-generating device according to either one of Ex71 or Ex72, in which the control electronics are configured to: maintain the second lighting array in a deactivated state over the first usage session; and maintain the first lighting array in a deactivated state over the second usage session.

Example Ex74: An aerosol-generating device according to any one of Ex66 to Ex73, in which the first and second lighting arrays are arranged parallel to each other; wherein the control electronics are configured to: activate all or a major portion of both the first lighting array and the second lighting array at the start of the usage session; progressively deactivate the first and second lighting arrays in synchronization with each other so as to progressively reduce an activated length of each of the first and second lighting arrays with progression through the usage session, such that the respective activated lengths of the first and second lighting arrays remain equal during the usage session.

Example Ex75: An aerosol-generating device according to any one of Ex66 to Ex74, in which the first and second lighting arrays are arranged parallel to each other in a collective arrangement, in which the collective arrangement of the first and second lighting arrays has a length and a width; wherein the control electronics are configured to: activate all or a major portion of both the first lighting array and the second lighting array at the start of the usage session; progressively deactivate the first and second lighting arrays in a snakewise manner across the width and along the length of the collective arrangement so as to progressively reduce an activated length of each of the first and second lighting arrays with progression through the usage session.

Example Ex76: An aerosol-generating device according to any one of Ex66 to Ex75, further comprising a substantially linear third lighting array located between and parallel to each of the first and second lighting arrays, in which the control electronics are configured to: activate all or a major portion of the third lighting array at the start of the usage session; progressively deactivate the third lighting array so as to progressively reduce an activated length of the third lighting array with progression through the usage session.

Example Ex77: An aerosol-generating device according to Ex76, in which the control electronics are configured such that on completion of the usage session, no light is emitted from the third lighting array.

Example Ex78: An aerosol-generating device according to either one of Ex76 or Ex77, in which the control electronics are configured to maintain the first and second lighting arrays in a deactivated state over the usage session.

Example Ex79: An aerosol-generating device according to any one of Ex1 to Ex78, the aerosol-generating device further comprising: a power source coupled to the control electronics; in which the aerosol-generating device is configured to receive an aerosol-generating article comprising the aerosol-forming substrate; wherein the control electronics are configured to: detect the receiving of the aerosol-generating article by the aerosol-generating device; control a supply of energy from the power source to an electrical heating arrangement to activate a pre-heating phase in which the electrical heating arrangement is heated to a predetermined target temperature; activate either or both of the first and second lighting arrays to generate a pre-heating light emission, in which the pre-heating light emission varies according to progression through the pre-heating phase so as to be indicative of progression through the pre-heating phase.

Example Ex80: An aerosol-generating device according to Ex79, in which: the aerosol-generating device comprises a cavity for receiving the aerosol-generating article, the aerosol-generating article comprising an inductively heatable susceptor; the electrical heating arrangement comprises an inductive heating arrangement coupled to the power source and configured to generate an alternating magnetic field within the cavity for inductively heating the susceptor of the aerosol-generating article when the aerosol-generating article is received in the cavity; the control circuitry configured to: generate probe power pulses for intermittently powering on the inductive heating arrangement; and detect a change of at least one property of the inductive heating arrangement due to the presence of the susceptor when an aerosol-generating article is received in the cavity, thereby enabling detection of the aerosol-generating article being received in the cavity.

Example Ex81: An aerosol-generating device according to Ex80, in which the at least one property is an equivalent resistance of the inductive heating arrangement or an inductance of the inductive heating arrangement.

Example Ex82: An aerosol-generating device according to any one of Ex79 to Ex81, in which the control electronics are configured to activate the first and second lighting arrays so that different portions of each of the first and second lighting arrays vary in luminance with time and with respect to each other, and the luminance of each of the first and second lighting arrays progressively increases over the pre-heating phase.

Example Ex83: An aerosol-generating device according to any one of Ex79 to Ex82, in which the control electronics are configured to activate the first and second lighting arrays such that a dominant wavelength of the predetermined emission of light progressively increases over the pre-heating phase.

Example Ex84: An aerosol-generating device according to any one of Ex79 to Ex83, in which the first and second lighting arrays are arranged parallel to each other; wherein the control electronics are configured to: activate the first and second lighting arrays in synchronization with each other so as to progressively increase an activated length of each of the first and second lighting arrays with progression through the pre-heating phase, such that the respective activated lengths of the first and second lighting arrays remain equal during the usage session.

Example Ex85: An aerosol-generating device according to any one of Ex79 to Ex84, in which the first and second lighting arrays are arranged parallel to each other in a collective arrangement, in which the collective arrangement has a length and a width; wherein the control electronics are configured to: activate the first and second lighting arrays in a snakewise manner across the width and along the length of the collective arrangement so as to progressively increase an activated length of each of the first and second lighting arrays with progression through the pre-heating phase.

Example Ex86: An aerosol-generating device according to any one of Ex79 to Ex85, in which the control electronics are configured to activate the first and second lighting arrays such that during or upon completion of the pre-heating phase the luminance of respective activated lengths of the first and second lighting arrays progressively increase with distance between first and second opposed ends of the respective activated length.

Example Ex87: An aerosol-generating device according to any one of Ex79 to Ex86, in which the control electronics are configured to activate the first and second lighting arrays such that during or upon completion of the pre-heating phase a dominant wavelength of the pre-heating light emission progressively increases with distance between first and second opposed ends of respective activated lengths of the first and second lighting arrays.

Example Ex88: An aerosol-generating device according to Ex87, in which the dominant wavelength is within a range of 380 to 750 nanometres, such that during or upon completion of the pre-heating phase the first opposed end defines a blue colour for the pre-heating light emission and the second opposed end defines a red colour for the pre-heating light emission.

Example Ex89: An aerosol-generating device according to any one of Ex79 to Ex88, in which the control electronics are configured such that both of the first and second lighting arrays have a uniform luminance along the length of the respective lighting array upon completion of the pre-heating phase.

Examples will now be further described with reference to the figures, in which:

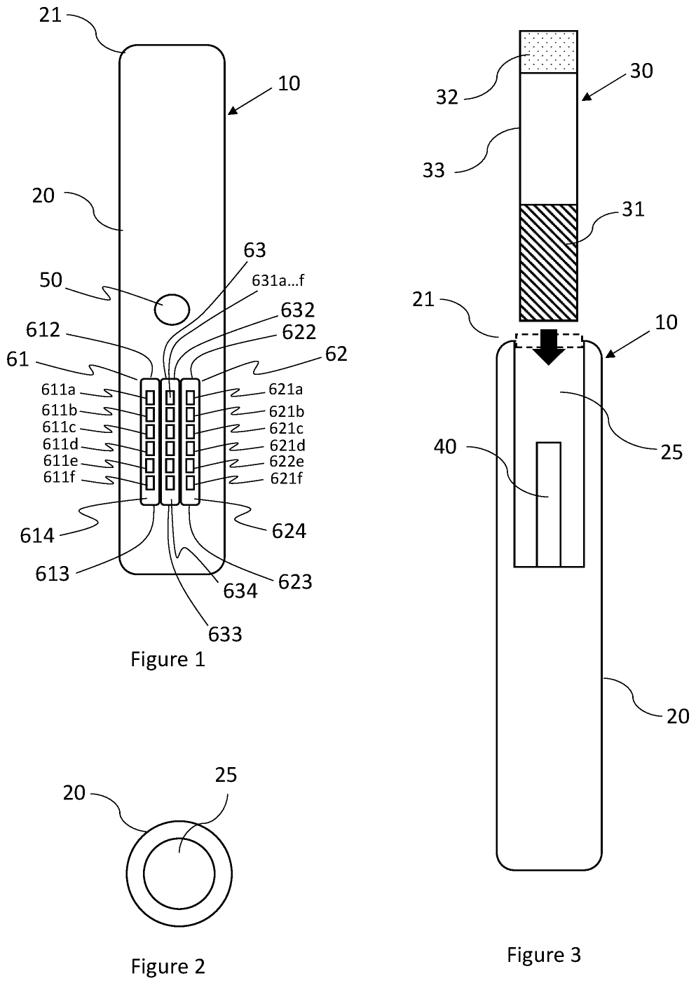

FIG. 1 illustrates a schematic side view of an aerosol-generating device;

FIG. 2 illustrates a schematic upper end view of the aerosol-generating device of FIG. 1;

FIG. 3 illustrates a schematic cross-sectional side view of the aerosol-generating device of FIG. 1 and an aerosol-generating article for use with the device;

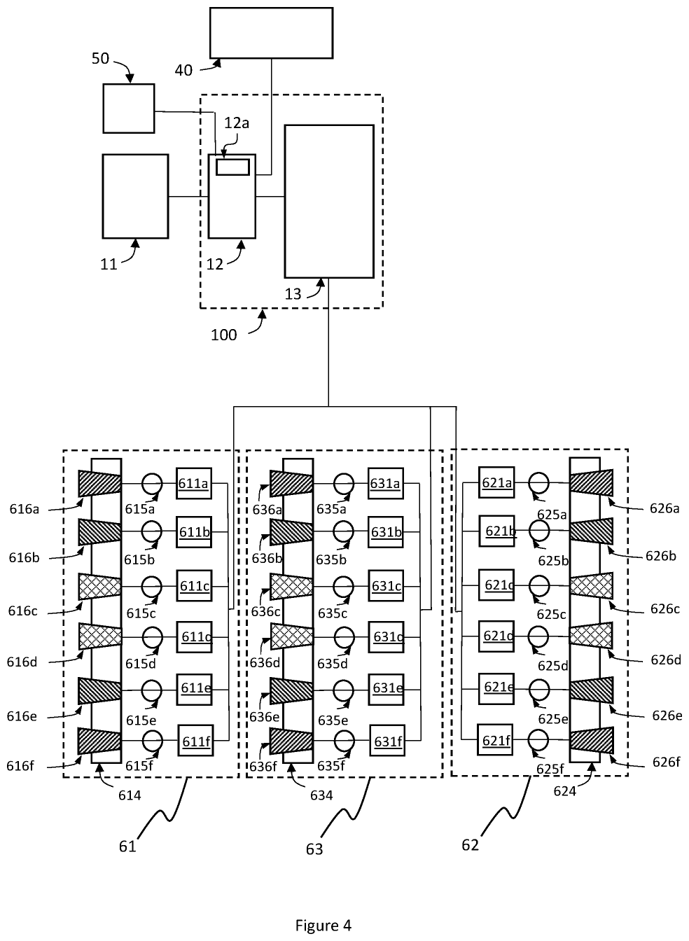

FIG. 4 is a block diagram providing a schematic illustration of various electronic components of the aerosol-generating device of FIGS. 1 to 3 and their interactions;

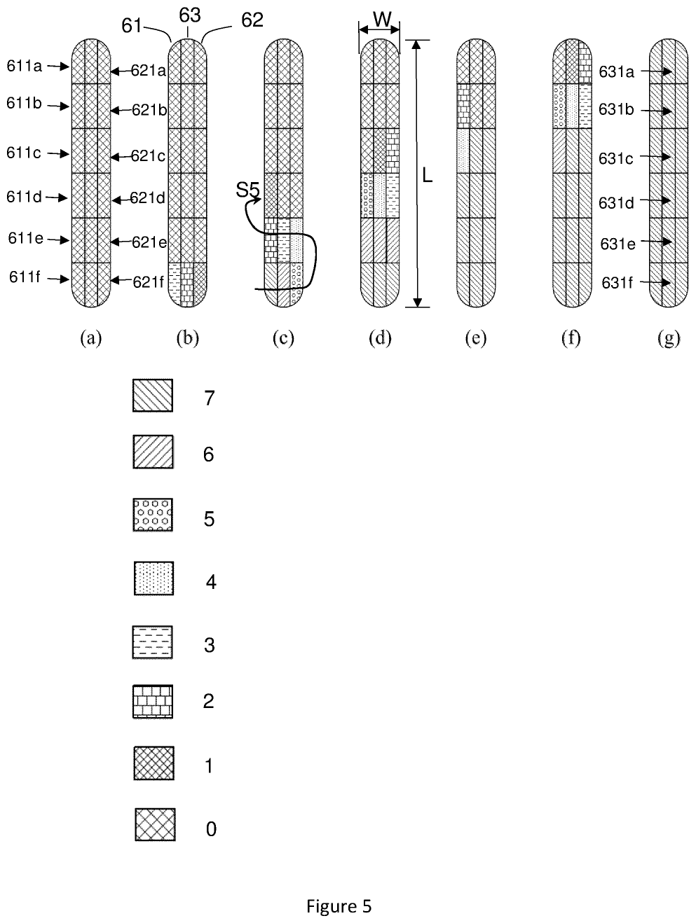

FIG. 5 illustrates an example of the operation of an arrangement of linear lighting arrays provided on the aerosol-generating device of FIGS. 1 to 4 with progression through a pre-heating phase of operation.

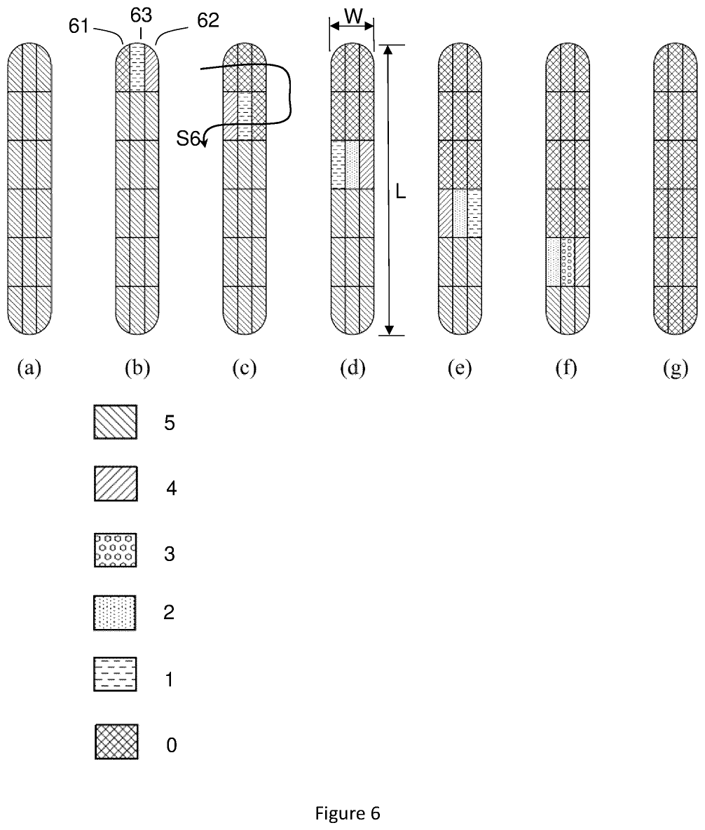

FIG. 6 illustrates an example of the operation of the arrangement of lighting arrays with progression through a usage session, the usage session commencing immediately after the pre-heating phase of operation illustrated in FIG. 5.

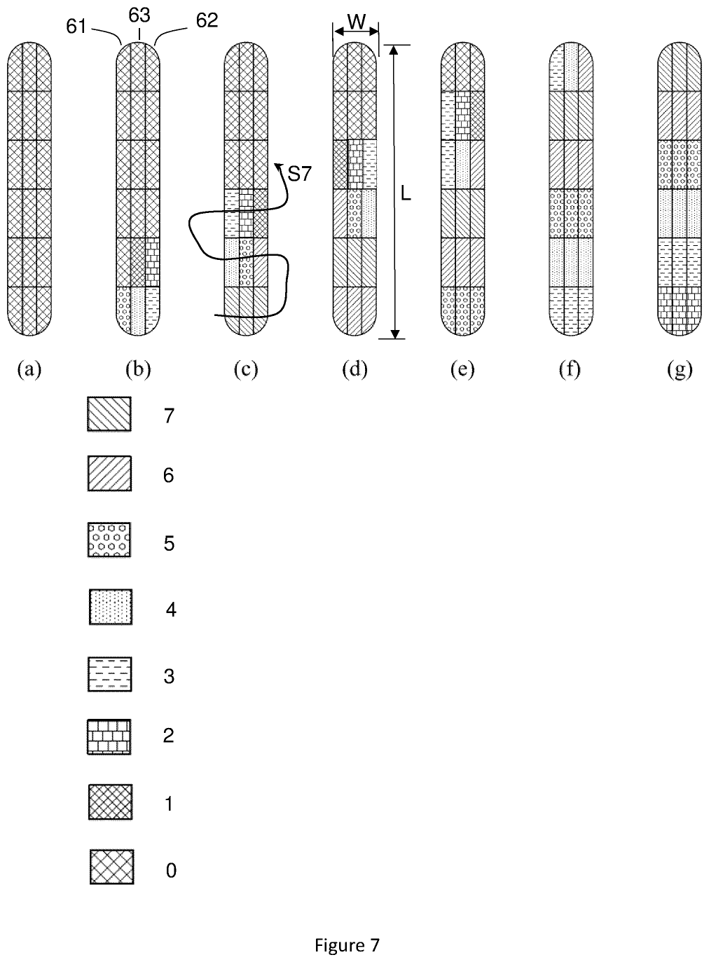

FIG. 7 illustrates a further example of the operation of the arrangement of lighting arrays with progression through a pre-heating phase of operation.

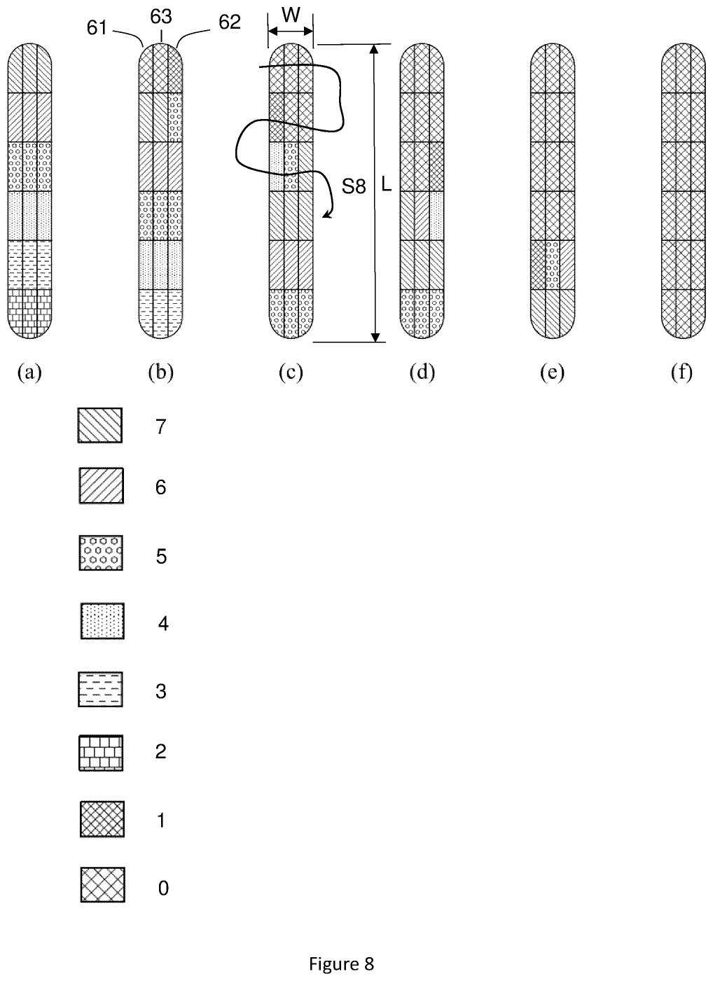

FIG. 8 illustrates a further example of the operation of the arrangement of lighting arrays with progression through a usage session, the usage session commencing immediately after the pre-heating phase of operation illustrated in FIG. 7.

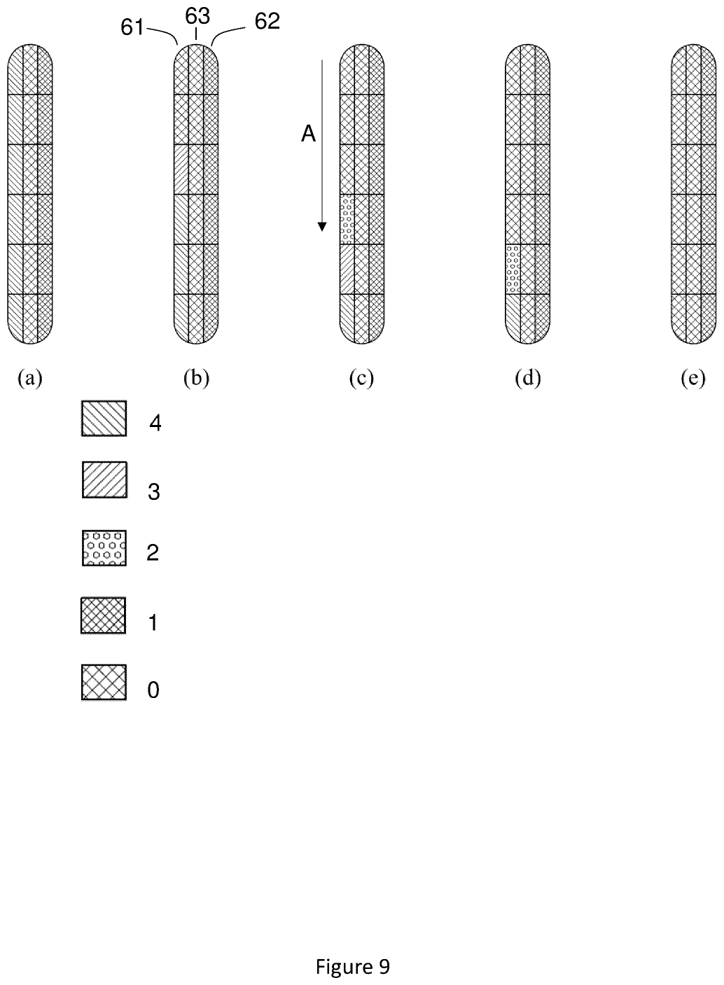

FIG. 9 illustrates an example of the operation of the arrangement of lighting arrays with progression through a first usage session.

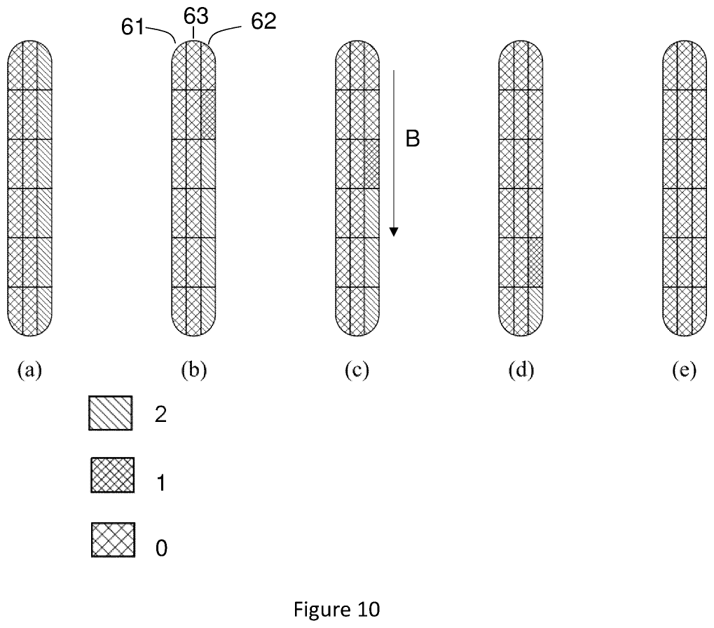

FIG. 10 illustrates an example of the operation of the arrangement of lighting arrays with progression through a second usage session, the second usage session following the first usage session illustrated in FIG. 9.

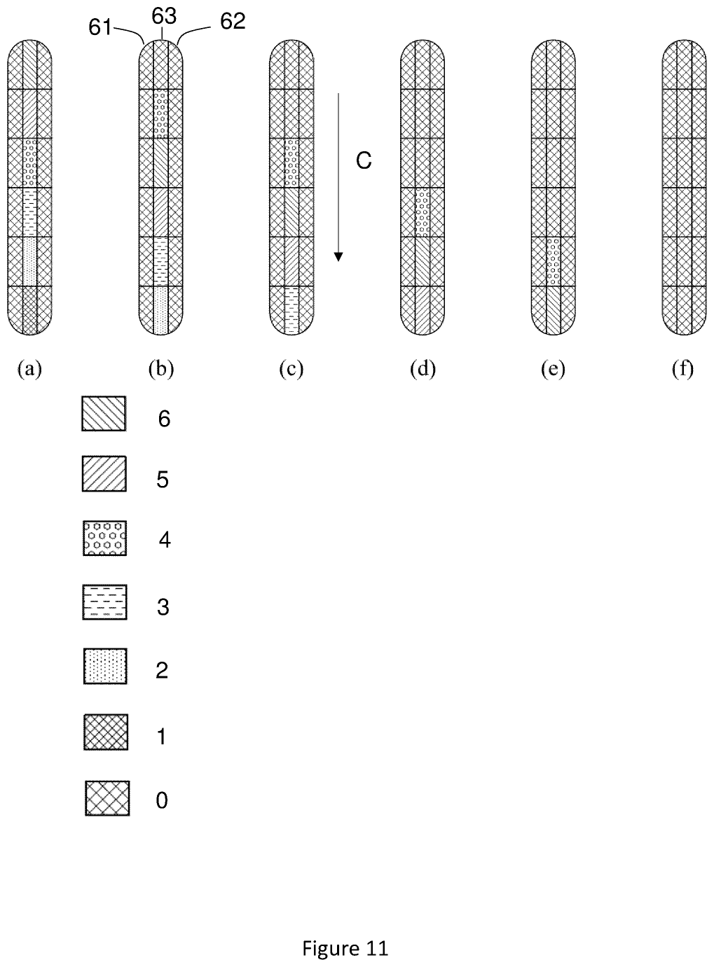

FIG. 11 illustrates a further example of the operation of the arrangement of lighting arrays with progression through a usage session.

An exemplary aerosol-generating device 10 is a hand-held aerosol generating device, and has an elongate shape defined by a housing 20 that is substantially circularly cylindrical in form (see FIGS. 1 and 2). As shown in FIGS. 2 and 3, the aerosol-generating device 10 comprises an open cavity 25 located at a proximal end 21 of the housing 20 for receiving an aerosol-generating article 30. Additionally, the aerosol-generating device 10 further has an electrically operated heater element 40 arranged to heat at least an aerosol-forming substrate 31 of the aerosol-generating article 30 when the aerosol-generating article is received in the cavity 25 (see FIG. 3).

The aerosol-generating device is configured to receive the aerosol-generating article 30. As shown in FIG. 3, the aerosol-generating article 30 has the form of a cylindrical rod, the rod formed by a combination of the aerosol-forming substrate 31 and a filter element 32. The aerosol-forming substrate 31 and filter element 32 are co-axially aligned and enclosed in a wrapper 33 of cigarette paper. The aerosol-forming substrate 31 is a solid aerosol-forming substrate comprising tobacco. However, in alternative embodiments (not shown), the aerosol-forming substrate 31 may instead be a liquid aerosol-forming substrate or formed of a combination of liquid and solid aerosol-forming substrates. The filter element 32 serves as a mouthpiece of the aerosol-generating article 30. The aerosol-generating article 30 has a diameter substantially equal to the diameter of the cavity 25 of the device 10 and a length longer than a depth of the cavity 25. When the aerosol-generating article 30 is received in the cavity 25 of the device 10, the portion of the article containing the filter element 32 extends outside of the cavity and may be drawn on by a user, in a similar manner to a conventional cigarette.

An arrangement of three linear lighting arrays 61, 62, 63 is incorporated into the housing 20 of the aerosol-generating device 10 (see FIG. 1). The arrangement consists of a first linear lighting array 61 and a second linear lighting array 62 which are arranged on either side of a third linear lighting array 63. So, the third linear lighting array 63 is centrally located between the first and second lighting arrays 61, 62. Each lighting array 61, 62, 63 is formed of a linear arrangement of six light emitting diodes 611a . . . f, 621a . . . f, 631a . . . f extending between a first end 612, 622, 632 and a second end 613, 623, 633 of the respective lighting array. All three lighting arrays 61, 62, 63 have the same length and are arranged parallel to one another, with their respective first and second ends aligned with one another. Each lighting array 61, 62, 63 has a respective display window 614, 624, 634 which forms part of the exterior surface of the housing 20 and is transparent to light. As will be described in more detail below, in use, light generated by the light emitting diodes of each lighting array 61, 62, 63 is directed towards the respective display window 614, 624, 634 of the respective lighting array so as to be visible to a user of the aerosol-generating device 10.

A battery 11 and microcontroller 12 are coupled to each other and located within the housing 20 (see FIG. 4). The microcontroller 12 also incorporates a memory module 12a. The microcontroller 12 is in turn coupled to both the heater element 40 and a lighting control driver 13. The microcontroller 12 and lighting control driver 13 collectively form a control electronics section 100 of the aerosol-generating device 10. The lighting control driver 13 is coupled to each of the light emitting diodes 611a . . . f, 621a . . . f, 631a . . . f of each lighting array 61, 62, 63. For the first lighting array 61, waveguides 615a . . . f are provided between the light emitting diodes 611a . . . f and the display window 614. Similarly, for the second lighting array 62, waveguides 625a . . . f are provided between the light emitting diodes 621a . . . f and the display window 624. For the third lighting array 63, waveguides 635a . . . f are also provided between the light emitting diodes 631a . . . f and the display window 634. Each one of the waveguides 615a . . . f, 625a . . . f, 635a . . . f is associated with a respective one of the light emitting diodes 611a . . . f, 621a . . . f, 631a . . . f of the respective lighting array 61, 62, 63, so that in use, each waveguide functions to direct light generated by an associated one of the light emitting diodes to the respective display window 614, 624, 634. The waveguides 615a . . . f, 625a . . . f, 635a . . . f are in the form of discrete lengths of optical fibre.

The memory module 12a contains instructions for execution by the microcontroller 12 and lighting control driver 13 during use of the device 10. The instructions stored in the memory module 12a include data on two or more user-selectable predetermined thermal profiles for the heater element 40, criteria determining the duration of a usage session, plus other data and information relevant to control and operation of the aerosol-generating device 10. When activated, the microcontroller 12 accesses the instructions contained in the memory module 12a and controls a supply of energy from the battery 11 to the heater element 40 according to the instructions contained in the memory module 12a. The microcontroller 12 also controls a supply of energy to the lighting control driver 13. In turn, the lighting control driver 13 individually controls a supply of electricity to each of the light emitting diodes 611a . . . f, 621a . . . f, 631a . . . f of the lighting arrays 61, 62, 63 such that each light emitting diode emits light 616a . . . f, 626a . . . f, 636a . . . f at one of a plurality of discrete static luminance levels under the control of the lighting control driver (see FIG. 4). The light emitted by different lighting emitting diodes of the three linear lighting arrays 61, 62, 63 under the control of the lighting control driver 13 together forms a predetermined light emission. The three different forms of cross-hatching used in FIG. 4 for the light 616a . . . f, 626a . . . f, 636a . . . f generated by different ones of the light emitting diodes of the first second and third lighting arrays 61, 62, 63 represent three different static luminance levels.

For the point in time shown in FIG. 4, the luminance of each of the three lighting arrays 61, 62, 63 is symmetric about the centre of the respective lighting array, with each of the three lighting arrays having the same variation in luminance over the lighting array length. So, with reference to the first lighting array 61, the centrally located two light emitting diodes 611c, d are independently controlled by the lighting control driver 13 to emit light at a first predetermined static luminance level, the neighbouring light emitting diodes 611b, e independently controlled to emit light at a second predetermined static luminance level and the outermost light emitting diodes 611a, f independently controlled to emit light at a third predetermined static luminance level. The second and third lighting arrays 62, 63 are controlled by the lighting control driver 13 to exhibit the same variation in luminance over their length as for the first lighting array 61.

In use, a user first inserts the aerosol-generating article 30 into the cavity 25 of the aerosol-generating device 10 (as shown by the arrow in FIG. 3) and turns on the device 10 by pressing a user button 50 to activate the heater element 40 to start a usage session. The button 50 is electro-mechanically coupled to the microcontroller 12 (see FIG. 4). In the embodiment shown, the button 50 also serves as a means for the user to select a given one of the predetermined thermal profiles stored in the memory module 12a. For the embodiment shown, a double-press of the button 50 functions to select a first predetermined thermal profile and a triple-press of the button functions to select a second predetermined thermal profile. However, in alternative embodiments (not shown), an alternative user interface may be provided with which a user can interact to select a desired one of the first and second predetermined thermal profiles. Such an alternative user interface may be in the form of a touch sensitive panel with which a user may engage a finger to select a desired one of the predetermined thermal profiles, the touch sensitive panel coupled to the microcontroller 12. Alternatively, the alternative user interface may include a motion or orientation sensor coupled to the microcontroller 12, in which a motion or gesture of the device 10 in a predetermined manner is detected by the sensor and serves as a means of selecting a specific one of the predetermined thermal profiles. The first and second predetermined thermal profiles differ from each other in their intensity, with the second predetermined thermal profile having a greater intensity than the first predetermined thermal profile. The second predetermined thermal profile is associated with supply of a greater amount of energy from the battery 11 to the heater element 40 over the usage session than for the first predetermined thermal profile.

After activation, the temperature of the heater element 40 is increased in a pre-heating phase from an ambient temperature to a predetermined target temperature for heating the aerosol-forming substrate 31 according to the selected predetermined thermal profile. On attainment of the predetermined target temperature, the usage session commences. Over the usage session, the heater element 40 heats the aerosol-forming substrate 31 of the article 30 such that volatile compounds of the aerosol-forming substrate are released and atomised to form an aerosol. The user draws on the filter element 32 of the article 30 and inhales the aerosol generated from the heated aerosol-forming substrate 31. The microcontroller 12 is configured to control the supply of energy from the battery 11 to maintain the heater element 40 at an approximately constant level as a user puffs on the article 30. The heater element 40 continues to heat the aerosol-generating article 30 in accordance with the selected predetermined thermal profile until an end of the usage session. At the end of the usage session, the heater element 40 is deactivated and allowed to cool. The usage session has a maximum duration defined by the first to occur of i) 6 minutes elapsing from activation of the heater element 40, or ii) the application by a user of 12 consecutive puffs to the aerosol-generating article 30. In an alternative embodiment, the maximum duration of the usage session is instead defined by the first to occur of i) 6 minutes elapsing from activation of the heater element 40, or ii) a cumulative volume of aerosol evolved from the aerosol-forming substrate over the usage session reaching a predetermined volume. In the illustrated embodiment, the heater element 40 is a resistance heater element. However, in other embodiments (not shown), the heater element 40 is instead in the form of a susceptor arranged within a fluctuating magnetic field such that it is heated by induction.

At the end of the usage session, the aerosol-generating article 30 is removed from the device 10 for disposal, and the device may be coupled to an external power source for charging of the battery 11 of the device.

FIG. 5 illustrates an example of how the lighting control driver 13 controls a supply of electricity from the battery 11 to individual ones of the light emitting diodes 611a . . . f, 621a . . . f, 631a . . . f of the first, second and third lighting arrays 61, 62, 63 to generate a predetermined light emission indicative of progression through a pre-heating phase of operation of the aerosol-generating device 10. The first, second and third lighting arrays form a collective lighting arrangement having a length L and a width W. As shown in the legend for FIG. 5, the light emitting diodes of each of the lighting arrays 61, 62, 63 are controlled by the lighting control driver 13 over the pre-heating phase to emit light having one of seven predetermined static luminance levels, or to be in a deactivated state in which no light is emitted. The light collectively generated by the three lighting arrays 61, 62, 63 at a given point in time in the pre-heating phase defines the predetermined light emission indicative of progression through the pre-heating phase of operation. The predetermined static luminance levels are designated as levels 7, 6, 5, 4, 3, 2 and 1, in order of decreasing luminance; the deactivated or “off” state is designated as level 0. At the start of the pre-heating phase of operation, the lighting control driver 13 maintains the lighting emitting diodes of all three lighting arrays 61, 62, 63 in a deactivated state in which no light is emitted, i.e. at level 0 (see FIG. 5 (a)). With progression through the pre-heating phase, the lighting control driver 13 controls the supply of electricity to the light emitting diodes 611a . . . f, 621a . . . f, 631a . . . f of the lighting arrays 61, 62, 63 to progressively activate the lighting arrays in a snakewise manner (see arrow S5) across the width W and along the length L of the collective lighting arrangement defined by the three lighting arrays. Over the course of the pre-heating phase, the lighting control driver 13 progressively increases the static luminance level of an increasing number of the light emitting diodes of the collective lighting arrangement formed by the three lighting arrays 61, 62, 63 from level 0 to level 7. As shown in FIG. 5 (g), at the end of the pre-heating phase, which is indicative of the heater element 40 having attained a predetermined target operating temperature, all of the light emitting diodes of all three lighting arrays are activated to emit light at their maximum static luminance level, i.e. level 7.

FIG. 6 illustrates an example of how the lighting control driver 13 controls a supply of electricity to individual ones of the light emitting diodes 611a . . . f, 621a . . . f, 631a . . . f of the first, second and third lighting arrays 61, 62, 63 to generate a predetermined light emission indicative of progression through the usage session. The usage session illustrated in FIG. 6 commences immediately after completion of the pre-heating phase of operation represented by FIG. 5. As shown in the legend for FIG. 6, the light emitting diodes of each of the lighting arrays 61, 62, 63 are controlled by the lighting control driver 13 over the usage session to emit light having one of five predetermined static luminance levels, or to be in a deactivated state in which no light is emitted. The light generated collectively by the three lighting arrays 61, 62, 63 at a given point in time in the usage session defines the predetermined light emission indicative of progression through the usage session. The predetermined static luminance levels are designated as levels 5, 4, 3, 2 and 1 order of decreasing luminance, with the deactivated or “off” state designated as level 0. At the start of the usage session, the lighting control driver 13 maintains the lighting emitting diodes 611a . . . f, 621a . . . f, 631a . . . f of all three lighting arrays 61, 62, 63 in a fully activated state in which all of the light emitting diodes generate light at the maximum static luminance level, i.e. level 5 (see FIG. 6 (a)). With progression through the usage session, the lighting control driver 13 controls the supply of electricity to the light emitting diodes 611a . . . f, 621a . . . f, 631a . . . f of the first, second and third lighting arrays 61, 62, 63 to progressively deactivate the lighting arrays in a snakewise manner (see arrow S6) across the width W and along the length L of the collective lighting arrangement defined by the three lighting arrays. Over the course of the usage session, the lighting control driver 13 progressively decreases the static luminance level of an increasing number of the light emitting diodes of the collective lighting arrangement formed by the three lighting arrays 61, 62, 63 from level 5 to level 0. As shown in FIG. 6 (g), at the end of the usage session all of the light emitting diodes of all three lighting arrays are in a deactivated state in which no light is emitted, i.e. in level 0.

FIG. 7 illustrates a further example of how the lighting control driver 13 controls a supply of electricity to individual ones of the light emitting diodes 611a . . . f, 621a . . . f, 631a . . . f of the first, second and third lighting arrays 61, 62, 63 to generate a predetermined light emission indicative of progression through the pre-heating phase of operation of the aerosol-generating device 10. As shown in the legend for FIG. 7, the light emitting diodes of each of the lighting arrays 61, 62, 63 are controlled by the lighting control driver 13 over the pre-heating phase to emit light having one of seven predetermined static luminance levels, or to be in a deactivated state in which no light is emitted. The light generated collectively by the three lighting arrays 61, 62, 63 at a given point in time in the pre-heating phase defines the predetermined light emission indicative of progression through the pre-heating phase of operation. The predetermined static luminance levels are designated as levels 7, 6, 5, 4, 3, 2 and 1 in order of decreasing luminance, with the deactivated or “off” state designated as level 0. At the start of the pre-heating phase of operation, the lighting control driver 13 maintains the light emitting diodes of all three lighting arrays 61, 62, 63 in a deactivated state in which no light is emitted, i.e. at level 0. With progression through the pre-heating phase, the lighting control driver 13 controls the supply of electricity to the light emitting diodes 611a . . . f, 621a . . . f, 631a . . . f of the first, second and third lighting arrays 61, 62, 63 to progressively activate the lighting arrays in a snakewise manner (see arrow S7) across the width W and along the length L of the collective lighting arrangement defined by the three lighting arrays. Over the course of the pre-heating phase, the lighting control driver 13 progressively increases the static luminance level of different ones of the light emitting diodes of the collective lighting arrangement formed by the three lighting arrays 61, 62, 63 between the different static luminance levels 1 to 7. As can be seen from FIG. 7 (g), at the completion of the pre-heating phase the corresponding light emitting diodes (indicated by one of letters a . . . f) of each lighting array 61, 62, 63 each emit light at a common static luminance level. So, the corresponding light emitting diodes of each lighting array 61, 62, 63 each generate a band of light at a specific one of the seven static luminance levels 1 to 7. At the completion of the pre-heating phase, the collective lighting arrangement formed by the three lighting arrays 61, 62, 63 generates a predetermined light emission consisting of six bands of light, each band having a distinct static luminance level, with there being a progressive stepwise increase in the static luminance level when moving from one band to another over the length L of the collective lighting arrangement.

FIG. 8 illustrates a further example of how the lighting control driver 13 controls a supply of electricity to individual ones of the light emitting diodes 611a . . . f, 621a . . . f, 631a . . . f of the first, second and third lighting arrays 61, 62, 63 to generate a predetermined light emission indicative of progression through the usage session. The usage session illustrated in FIG. 8 commences immediately after completion of the pre-heating phase of operation represented by FIG. 7. As shown in the legend for FIG. 8, the light emitting diodes of each of the lighting arrays 61, 62, 63 are controlled by the lighting control driver 13 over the usage session to emit light having one of seven predetermined static luminance levels, or to be in a deactivated state in which no light is emitted. The light generated collectively by the three lighting arrays at a given point in time in the usage session defines the predetermined light emission indicative of progression through the usage session. The predetermined static luminance levels are designated as levels 7, 6, 5, 4, 3, 2 and 1 in order of decreasing luminance, with the deactivated or “off” state designated as level 0. At the start of the usage session (shown in FIG. 8 (a)), the lighting control driver 13 maintains the light emitting diodes 611a . . . f, 621a . . . f, 631a . . . f of all three lighting arrays 61, 62, 63 in the same banded state they were in at the completion of the pre-heating phase in FIG. 7 (g). With progression through the usage session, the lighting control driver 13 controls the supply of electricity to the light emitting diodes 611a . . . f, 621a . . . f, 631a . . . f of the first, second and third lighting arrays 61, 62, 63 to progressively deactivate the lighting arrays in a snakewise manner (see arrow S8) across the width W and along the length L of the collective lighting arrangement defined by the three lighting arrays. Over the course of the usage session, the lighting control driver 13 progressively decreases the static luminance level of an increasing number of the light emitting diodes of the collective lighting arrangement formed by the three lighting arrays 61, 62, 63 from level 7 to level 0. At the end of the usage session, all of the light emitting diodes of all three lighting arrays are in a deactivated state in which no light is emitted, i.e. in level 0.

FIG. 9 illustrates an example of how the lighting control driver 13 controls a supply of electricity to individual ones of the light emitting diodes 611a . . . f of the first lighting array 61 to generate a predetermined light emission indicative of progression through a first usage session. The battery 11 is provided in a fully charged state at the commencement of the first usage session. The legend for FIG. 9 shows four different predetermined static luminance levels of light, designated as levels 4, 3, 2 and 1, plus a deactivated or “off” state, designated as level 0. At the commencement of the first usage session, all of the light emitting diodes 611a . . . f of the first lighting array 61 are controlled by the lighting control driver 13 to emit light at the peak static luminance level, i.e. level 4. With progression through the first usage session, the lighting control driver 13 progressively reduces an activated length of the first lighting array 61 by progressively reducing the static luminance level of the light emitting diodes 611a . . . f from level 4, to level 3, to level 2 to the deactivated state. The reduction in the static luminance level commences with light emitting diode 611a and progresses downwards in the direction of arrow A along the length of the first lighting array 61 to each of the successive light emitting diodes of the lighting array 61. On completion of the first usage session, all of the light emitting diodes 611a . . . f of the first lighting array 61 are in the deactivated state, i.e. in level 0. Over the entire first usage session, all of the light emitting diodes 621a . . . f are controlled by the lighting control driver to emit light with a minimal static luminance level of level 1. The central or third lighting array 63 is controlled by the lighting control driver 13 to be maintained in a deactivated state over the first usage session.

FIG. 10 illustrates an example of how the lighting control driver 13 controls a supply of electricity to individual ones of the light emitting diodes 621a . . . f of the second lighting array 62 to generate a predetermined light emission indicative of progression through a second usage session. The second usage session follows the first usage session, with the aerosol-generating device 10 being powered over the second usage session using whatever energy remains in the battery 11 after the first usage session. The legend for FIG. 10 shows two different predetermined static luminance levels of light, designated as levels 2 and 1, plus a deactivated or “off” state, designated as level 0. At the commencement of the second usage session, all of the light emitting diodes 621a . . . f of the second lighting array 62 are controlled by the lighting control driver 13 to emit light at the peak static luminance level, i.e. level 2. With progression through the second usage session, the lighting control driver 13 progressively reduces an activated length of the second lighting array 62 by progressively reducing the static luminance level of the light emitting diodes 621a . . . f from level 2, to level 1, to the deactivated state. The reduction in the static luminance level commences with light emitting diode 621a and progresses downwards in the direction of arrow B along the length of the second lighting array 62 to each of the successive light emitting diodes of the lighting array 62. On completion of the usage session, all of the light emitting diodes 621a . . . f of the second lighting array 62 are in the deactivated state, i.e. in level 0. Over the entire second usage session, all of the light emitting diodes 611a . . . f, 631a . . . f of the first and third lighting arrays 61, 63 are controlled by the lighting control driver 13 to be maintained in a deactivated state in which no light is emitted.

FIG. 11 illustrates an example of how the lighting control driver 13 controls a supply of electricity to individual ones of the light emitting diodes 631a . . . f of the central or third lighting array 63 to generate a predetermined light emission indicative of progression through a usage session. The legend for FIG. 11 shows six different predetermined static luminance levels of light, designated as levels 6, 5, 4, 3, 2 and 1, plus a deactivated or “off” state, designated as level 0. At the commencement of the usage session, all of the light emitting diodes 631a . . . f of the third lighting array 63 are controlled by the lighting control driver 13 to define a progressive reduction in luminance between light emitting diode 631a and light emitting diode 631f. When moving downwards along the length of the lighting array 63, each successive light emitting diode of the lighting array 63 emits light at a lower one of the predetermined static luminance levels than its predecessor. With progression through the usage session, the lighting control driver 13 progressively reduces an activated length of the third lighting array 63 by progressively reducing the static luminance level of the light emitting diodes 631a . . . f from level 6 to the deactivated state. The reduction in the static luminance level commences with light emitting diode 631a and progresses downwards in the direction of arrow C along the length of the third lighting array 63 to each of the successive light emitting diodes of the lighting array 63. On completion of the usage session, all of the light emitting diodes 631a . . . f of the third lighting array 63 are in the deactivated state, i.e. in level 0. Over the entire usage session, all of the light emitting diodes of the first and second lighting arrays 61, 62 are maintained by the lighting control driver 13 in a deactivated state.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number “A” is understood as “A”±10% of “A”. Within this context, a number “A” may be considered to include numerical values that are within general standard error for the measurement of the property that the number “A” modifies. The number “A”, in some instances as used in the appended claims, may deviate by the percentages enumerated above provided that the amount by which “A” deviates does not materially affect the basic and novel characteristic(s) of the claimed invention. Also, all ranges include the maximum and minimum points disclosed and include any intermediate ranges therein, which may or may not be specifically enumerated herein.

1.-15. (canceled)

16. An aerosol-generating device for heating an aerosol-forming substrate to generate an inhalable aerosol during a usage session, the aerosol-generating device comprising:
control electronics; and
a substantially linear first lighting array and a substantially linear second lighting array, each of the first and the second lighting arrays extending over a length between first and second ends of the respective lighting array,
wherein the first and the second lighting arrays are each of a same length and laterally spaced apart from each other so as to be parallel and aligned with each other, and the first and the second ends of the first lighting array are aligned with the first and the second ends of the second lighting array,
wherein the control electronics are coupled to the first and the second lighting arrays and configured to activate both of the first and the second lighting arrays to generate a predetermined light emission indicative of and in response to at least one of:
i) a status of the aerosol-generating device, and
ii) progression of an operational phase of the aerosol-generating device, and
wherein the control electronics are further configured to cause each of the first and the second lighting arrays to convey different data to a user.

17. The aerosol-generating device according to claim 16, wherein the control electronics are further configured to activate either or both of the first and the second lighting arrays at two or more luminance levels, so as to control a luminance of the predetermined light emission.

18. The aerosol-generating device according to claim 16, wherein the control electronics are further configured to activate either or both of the first and the second lighting arrays in two or more colour states, so as to control a colour of the predetermined light emission.

19. The aerosol-generating device according to claim 16, wherein the control electronics are further configured to activate either or both of the first and the second lighting arrays so as to vary the predetermined light emission with respect to time.

20. The aerosol-generating device according to claim 19, wherein the control electronics are further configured to activate either or both of first and the second lighting arrays to vary the predetermined light emission with respect to time so as to be indicative of a progression of the operational phase of the aerosol-generating device.

21. The aerosol-generating device according to claim 20, wherein the progression of the operational phase is a progression of the usage session.

22. The aerosol-generating device according to claim 19, wherein the control electronics are further configured to activate either or both of the first and the second lighting arrays so as to vary an activated length of the respective lighting array with respect to time.

23. The aerosol-generating device according to claim 19, wherein the control electronics are further configured to vary the predetermined light emission with respect to time in one or more of luminance and colour.

24. The aerosol-generating device according to claim 19, wherein the control electronics are further configured to vary the predetermined lighting emission with respect to time by one or more of activating, deactivating, and reactivating different portions of either or both of the first and the second lighting arrays over time.

25. The aerosol-generating device according to claim 16, wherein each of the first and second lighting arrays comprise a plurality of light emitting units distributed between the first and the second ends of the respective lighting array.

26. The aerosol-generating device according to claim 25, further comprising one or more waveguides configured to direct light generated by one or more of the plurality of light emitting units to one or more display windows for viewing of the predetermined light emission by the user.

27. The aerosol-generating device according to claim 25, wherein each one of the plurality of light emitting units comprises a light emitting diode and the control electronics comprises a light emitting diode control driver and a separate microcontroller, the light emitting diode control driver being configured to control a supply of electricity from a power source to one or more of the plurality of light emitting diodes of either or both of the first and the second lighting arrays under control of the microcontroller, so as to generate the predetermined light emission.

28. The aerosol-generating device according to claim 27,
wherein the plurality of light emitting units of each of the first and the second lighting arrays comprises:
a first set of one or more light emitting diodes configured to emit light of a first colour, and
a second set of one or more light emitting diodes configured to emit light of a second colour, and
wherein the light emitting diode control driver is further configured to activate one or more of the light emitting diodes from the first set alone of either or both of the first and the second lighting arrays, or from the second set alone of either or both of the first and the second lighting arrays, or from both of the first and the second sets of either or both of the first and the second lighting arrays, so as to control a colour of the predetermined light emission.

29. The aerosol-generating device according to claim 16,
further comprising a substantially linear third lighting array located between and parallel to each of the first and the second lighting arrays,
wherein the control electronics are further configured to activate the third lighting array alone or in addition to either or both of the first and the second lighting array to generate the predetermined light emission.

30. The aerosol-generating device according to claim 16, wherein the predetermined light emission is one or more of a usage session light emission, a low energy light emission, a thermal profile light emission, a pause light emission, a state change light emission, a progressing light emission, and a pre-heating light emission.
